(19) United States
(12) Patent Application Publication
(10) Pub. No.: US 2026/0166163 A1
(43) Pub. Date: Jun. 18, 2026

(54) METHODS FOR TREATING CANCER

(71) Applicant: BicycleTx Limited, Cambridge (GB)

(72) Inventors: Shawn Watson, Cambridge (GB); Dominic Smethurst, Cambridge (GB); Punit Upadhyaya, Cambridge (GB); David Witty, Cambridge (GB); Kevin McDonnell, Cambridge (GB)

(21) Appl. No.: 18/710,083

(22) PCT Filed: Nov. 16, 2022
(86) PCT No.: PCT/GB2022/052903

§ 371 Date: May 14, 2024

Related U.S. Application Data
(60) Provisional application No. 63/264,132, filed on Nov. 16, 2021.

(51) Int. Cl. A61K 47/64; A61K 39/395; A61K 47/02; A61K 47/18; A61K 47/26; A61K 47/54; A61P 35/00

(52) U.S. Cl. CPC A61K 47/64; A61K 39/3955; A61K 47/02; A61K 47/18; A61K 47/26; A61K 47/545; A61P 35/00

(57) ABSTRACT

The present invention relates to a heterotandem bicyclic peptide complex BT7480, or pharmaceutically acceptable salts thereof, or pharmaceutical compositions thereof, and uses thereof.

### CROSS REFERENCE TO RELATED APPLICATIONS

This application claims priority to U.S. Provisional Application No. 63/264,132, filed Nov. 16, 2021, which is hereby incorporated herein by reference in its entirety.

### TECHNICAL FIELD OF THE INVENTION

The present invention relates to a heterotandem bicyclic peptide complex BT7480, or pharmaceutically acceptable salts thereof, or pharmaceutical compositions thereof. The present invention also provides uses of a heterotandem bicyclic peptide complex BT7480, or pharmaceutically acceptable salts thereof, or pharmaceutical compositions thereof, for preventing or treating a Nectin-4 associated advanced malignancy.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7(7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example TATA (1,1′,1″-(1,3,5-triazinane-1,3,5-triyl)triprop-2-en-1-one, Heinis et al. Angew Chem, Int Ed. 2014; 53:1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to one aspect, the invention provides a solid pharmaceutical composition comprising BT7480, or a pharmaceutically acceptable salt thereof, Tris base, mannitol, and sodium hydroxide. According to another aspect, the invention provides an aqueous pharmaceutical composition comprising BT7480, or a pharmaceutically acceptable salt thereof, Tris base, mannitol, sodium hydroxide, and water.

In another aspect, the invention provides a method for treating a Nectin-4 associated advanced malignancy in a patient comprising administering to the patient a pharmaceutical composition as described herein. In some embodiments, the invention provides a method for treating a Nectin-4 associated advanced malignancy in a patient comprising administering to the patient by IV infusion an aqueous pharmaceutical composition comprising BT7480, or a pharmaceutically acceptable salt thereof, Tris base, mannitol, sodium hydroxide, sodium chloride, and water.

### DETAILED DESCRIPTION OF CERTAIN EMBODIMENTS

### 1. General Description of Certain Embodiments of the Invention

In one aspect, the invention provides methods of using a pharmaceutical composition described herein for treating a Nectin-4 associated advanced malignancy.

In some embodiments, the invention provides a method for treating a Nectin-4 associated advanced malignancy in a patient comprising administering weekly by IV infusion to the patient an aqueous pharmaceutical composition comprising BT7480, or a pharmaceutically acceptable salt thereof, Tris base, mannitol, sodium hydroxide, sodium chloride, and water. In some embodiments, an aqueous pharmaceutical composition as described herein is administered in combination with nivolumab.

In some embodiments, a Nectin-4 associated advanced malignancy is selected from the group consisting of bladder cancer, esophageal cancer, non-small-cell lung cancer (NSCLC), head and neck cancer, ovarian cancer, breast cancer such as triple-negative breast cancer (TNBC), gastric/upper gastrointestinal (GI) cancer, melanoma, pancreatic cancer, and urothelial cancer.

In some embodiments, a Nectin-4 associated advanced malignancy is non-small-cell lung cancer (NSCLC). In some embodiments, a Nectin-4 associated advanced malignancy is ovarian cancer. In some embodiments, a Nectin-4 associated advanced malignancy is breast cancer such as triple-negative breast cancer (TNBC). In some embodiments, a Nectin-4 associated advanced malignancy is gastric/upper gastrointestinal (GI). In some embodiments, a Nectin-4 associated advanced malignancy is pancreatic cancer. In some embodiments, a Nectin-4 associated advanced malignancy is urothelial cancer. In some embodiments, a Nectin-4 associated advanced malignancy is bladder cancer. In some embodiments, a Nectin-4 associated advanced malignancy is head and neck cancer. In some embodiments, a Nectin-4 associated advanced malignancy is esophageal cancer. In some embodiments, a Nectin-4 associated advanced malignancy is melanoma.

### 2. Compounds and Definitions

The term “BT7480,” as used herein, is a heterotandem bicyclic peptide complex consisting of a Nectin-4 specific peptide linked to two CD137 specific peptides via a N-(acid-PEG₃)-N-bis(PEG₃-azide) linker, having a structure as shown below.

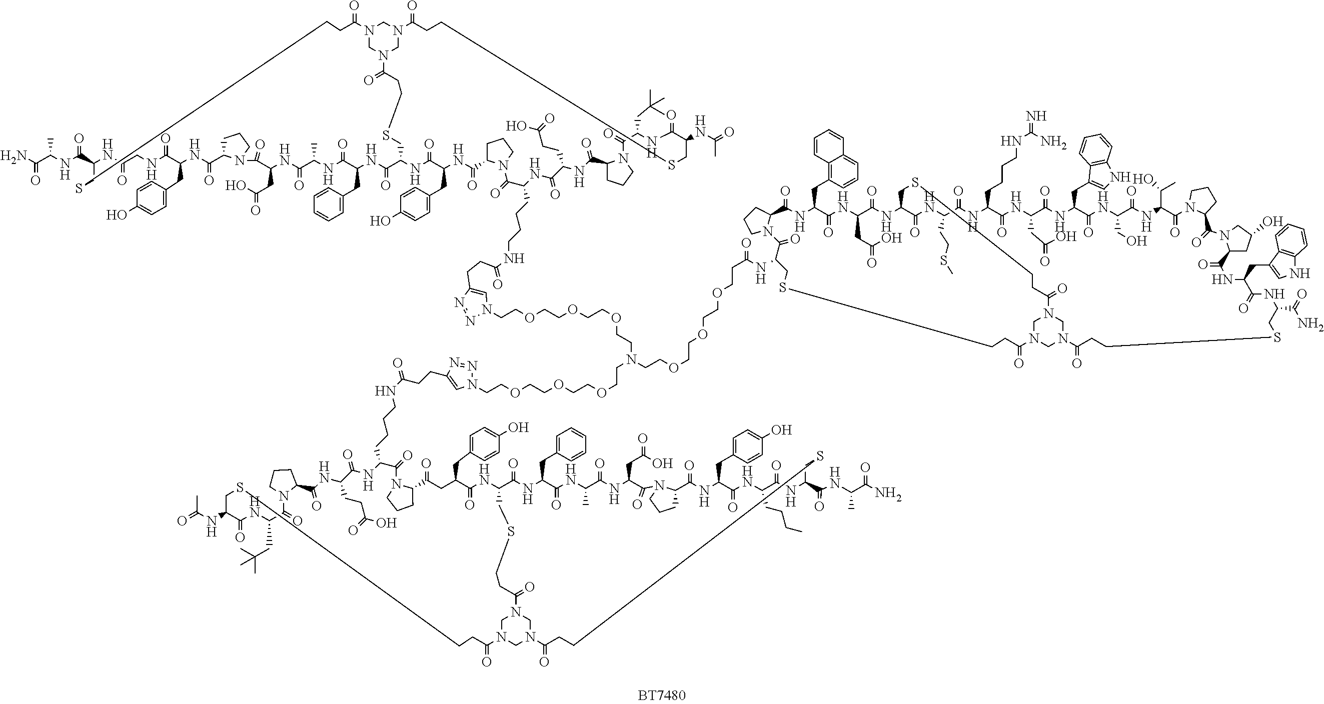

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. Mi Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

As used herein, the term “about” shall have the meaning of within 10% of a given value or range. In some embodiments, the term “about” refers to within 10%, 9%, 8%, 7%, 6%, 5%, 4%, 3%, 2%, or 1% of a given value.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Pharmaceutical Compositions

According to one aspect, the invention provides a solid pharmaceutical composition comprising BT7480, or a pharmaceutically acceptable salt thereof, Tris base, mannitol, and sodium hydroxide. In some embodiments, a solid pharmaceutical composition of the invention comprises about 66 mg BT7480, or a pharmaceutically acceptable salt thereof.

In some embodiments, a solid pharmaceutical composition of the invention is in powder form. In some embodiments, a solid pharmaceutical composition of the invention is a lyophilized powder. In some embodiments, a solid pharmaceutical composition of the invention is a sterile lyophilized powder.

In some embodiments, the invention provides a 4R injection vial containing a solid pharmaceutical composition as described herein. In some embodiments, the invention provides a 4R injection vial containing 66 mg BT7480, or a pharmaceutically acceptable salt thereof, Tris base, mannitol, and sodium hydroxide.

According to one aspect, the invention provides an aqueous pharmaceutical composition comprising BT7480, or a pharmaceutically acceptable salt thereof, Tris base, mannitol, sodium hydroxide, and water. In some embodiments, an aqueous pharmaceutical composition comprises BT7480 at a concentration of 60 mg/mL, Tris base, mannitol, sodium hydroxide, and water. In some embodiments, the invention provides a 4R injection vial containing an aqueous pharmaceutical composition as described herein. In some embodiments, the invention provides a 4R injection vial containing 1.1 mL aqueous pharmaceutical composition, comprising BT7480 at a concentration of 60 mg/mL, Tris base, mannitol, sodium hydroxide, and water.

In some embodiments, the invention provides an aqueous pharmaceutical composition comprising BT7480, or a pharmaceutically acceptable salt thereof, Tris base, mannitol, sodium hydroxide, sodium chloride, and water. In some embodiments, an aqueous pharmaceutical composition is prepared by diluting an aqueous pharmaceutical composition comprises BT7480 at a concentration of 60 mg/mL, Tris base, mannitol, sodium hydroxide, and water into 0.9% saline. In some embodiments, the invention provides an infusion bag containing BT7480, or a pharmaceutically acceptable salt thereof, Tris base, mannitol, sodium hydroxide, sodium chloride, and water.

In some embodiments, an aqueous pharmaceutical composition of the invention comprises an acceptable vehicle or solvent. In some embodiments, an acceptable vehicle or solvent is selected from sterile water, Ringer's solution, U.S.P. and isotonic sodium chloride solution. In some embodiments, an acceptable vehicle or solvent is sterile water. In some embodiments, an acceptable vehicle or solvent is a sterile injectable medium.

In some embodiments, a pharmaceutically acceptable excipient or carrier comprises a buffering agent. In some embodiments, a buffering agent is selected from phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, a buffering agent is histidine. In some embodiments, a buffering agent is sodium hydroxide. In some embodiments, a buffering agent is hydrochloric acid.

In some embodiments, an aqueous pharmaceutical composition of the invention is at a pH of about 6-8. In some embodiments, an aqueous pharmaceutical composition of the invention is at a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, 7.9, or about 8.0. In some embodiments, an aqueous pharmaceutical composition of the invention is at a pH of about 7.0 or about 7.5. In some embodiments, an aqueous pharmaceutical composition of the invention is at a pH of about 7.0-7.5.

In some embodiments, a pharmaceutically acceptable excipient or carrier comprises a stabilizer or cryoprotectant. In some embodiments, a stabilizer or cryoprotectant is dimethyl sulfoxide (DMSO). In some embodiments, a stabilizer or cryoprotectant is ethylene glycol. In some embodiments, a stabilizer or cryoprotectant is glycerol. In some embodiments, a stabilizer or cryoprotectant is propylene glycol. In some embodiments, a stabilizer or cryoprotectant is 2-methyl-2, 4-pentanediol (MPD). In some embodiments, a stabilizer or cryoprotectant is trehalose. In some embodiments, a stabilizer or cryoprotectant is formamide. In some embodiments, a stabilizer or cryoprotectant is proline. In some embodiments, a stabilizer or cryoprotectant is glycerol 3-phosphate. In some embodiments, a stabilizer or cryoprotectant is sorbitol. In some embodiments, a stabilizer or cryoprotectant is diethyl glycol. In some embodiments, a stabilizer or cryoprotectant is sucrose.

In some embodiments, a pharmaceutically acceptable excipient or carrier comprises an isotonicity adjusting agent. In some embodiments, an isotonicity adjusting agent is sodium chloride, dextrose, calcium chloride, or a combination thereof. In some embodiments, an isotonicity adjusting agent is dextrose. In some embodiments, an isotonicity adjusting agent is sodium chloride. In some embodiments, an isotonicity adjusting agent is a combination of sodium chloride and dextrose.

In some embodiments, the invention provides a solid pharmaceutical composition comprising:
BT7480, or a pharmaceutically acceptable salt thereof;
about 0.1 mg Tris base per mg of BT7480, or a pharmaceutically acceptable thereof;
about 0.4 mg mannitol per mg of BT7480, or a pharmaceutically acceptable thereof; and
sodium hydroxide, wherein the amount of sodium hydroxide provides a pH of about 7.0 to 7.5
when the solid pharmaceutical composition is reconstituted in water.

In some embodiments, the invention provides a solid pharmaceutical composition,
which is a lyophilized powder, comprising:
about 66 mg BT7480, or a pharmaceutically acceptable salt thereof;
about 6.6 mg Tris base;
about 26.4 mg mannitol; and
sodium hydroxide, wherein the amount of sodium hydroxide provides a pH of about 7.0 to 7.5
when the solid pharmaceutical composition is reconstituted in water.

In some embodiments, the invention provides an aqueous pharmaceutical composition comprising:
about 60 mg/mL BT7480, or a pharmaceutically acceptable salt thereof;
about 6 mg/mL Tris base;
about 24 mg/mL mannitol;
sodium hydroxide; and
water, wherein the aqueous pharmaceutical composition is at a pH of about 7.0 to 7.5.

In some embodiments, the invention provides an aqueous pharmaceutical composition at a volume of about 1.1 mL, comprising:
about 60 mg/mL BT7480, or a pharmaceutically acceptable salt thereof;
about 6 mg/mL Tris base;
about 24 mg/mL mannitol;
sodium hydroxide; and
water, wherein the aqueous pharmaceutical composition is at a pH of about 7.0 to 7.5.

In some embodiments, the invention provides an aqueous pharmaceutical composition prepared by reconstituting a solid pharmaceutical composition of the invention in water. In some embodiments, the invention provides an aqueous pharmaceutical composition prepared by dissolving a solid pharmaceutical composition of the invention in an injectable medium (e.g., 0.9% w/v saline). In some embodiments, the invention provides an aqueous pharmaceutical composition prepared by reconstituting a solid pharmaceutical composition of the invention in water, followed by dilution into 0.9% w/v saline. In some embodiments, an aqueous pharmaceutical composition is diluted into a 0.9% w/v saline IV bag for IV infusion.

### 4. Uses of the Pharmaceutical Compositions

In one aspect, the invention provides a method, or a use, for treating a Nectin-4 associated advanced malignancy in a patient comprising administering to the patient a pharmaceutical composition as described herein. In some embodiments, the invention provides a method, or a use, for treating a Nectin-4 associated advanced malignancy in a patient with renal insufficiency comprising administering to the patient a pharmaceutical composition as described herein. In some embodiments, renal insufficiency refers to CLcr>30 to 59 mL/min by the MDRD equation or local equivalent. In some embodiments, a method or a use as described here further comprises administering nivolumab to a patient.

In some embodiments, nivolumab is administered to a patient at a dosage level of 240 mg once every 2 weeks via an infusion of about 30 minutes. In some embodiments, nivolumab is diluted into 0.9% sodium chloride. In some embodiments, nivolumab is diluted into 5% dextrose. In some embodiments, nivolumab is diluted to a final concentration of about 1 mg/mL to about 10 mg/ml before infusion. In some embodiments, an aqueous pharmaceutical composition as described herein is administered in combination with nivolumab by sequential infusions first with BT7480 followed by nivolumab. In some embodiments, an aqueous pharmaceutical composition as described herein is administered about 1 hour before nivolumab is administered. In some embodiments, an aqueous pharmaceutical composition as described herein is administered about 10 minutes, about 20 minutes, about 30 minutes, about 40 minutes, or about 50 minutes before nivolumab is administered.

In some embodiments, a method of the invention comprises administering to a patient intravenously a pharmaceutical composition as described herein. In some embodiments, a pharmaceutical composition of the invention is administered by an IV injection. In some embodiments, a pharmaceutical composition of the invention is administered by an IV infusion. In some embodiments, an IV infusion of a pharmaceutical composition of the invention lasts about 5-30 minutes. In some embodiments, an IV infusion of a pharmaceutical composition of the invention lasts about 30-60 minutes. In some embodiments, an IV infusion of a pharmaceutical composition of the invention lasts about 55-75 minutes. In some embodiments, an IV infusion of a pharmaceutical composition of the invention lasts about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, or about 90 minutes. In some embodiments, an IV infusion of a pharmaceutical composition of the invention lasts about 60 minutes. In some embodiments, an IV infusion of a pharmaceutical composition of the invention lasts about 2 hours, about 2.5 hours, about 3 hours, about 3.5 hours, or about 4 hours.

In some embodiments, a pharmaceutical composition of the invention is administered to a patient once every 1, 2, 3, 4, 5, 6, or 7 days. In some embodiments, there are about 6 hours between two administrations of a pharmaceutical composition of the invention to a patient. In some embodiments, there are about 12 hours, about 18 hours, or about 24 hours between two administrations of a pharmaceutical composition of the invention to a patient. In some embodiments, there are about 36 hours, about 48 hours, about 60 hours, about 72 hours, about 84 hours, about 96 hours, or about 108 hours between two administrations of a pharmaceutical composition of the invention to a patient. In some embodiments, a pharmaceutical composition of the invention is administered to a patient once weekly. In some embodiments, a pharmaceutical composition of the invention is administered to a patient once every two weeks. In some embodiments, a pharmaceutical composition of the invention is administered to a patient once every three weeks. In some embodiments, a pharmaceutical composition of the invention is administered to a patient once every four weeks. In some embodiments, a pharmaceutical composition of the invention is administered to a patient monthly.

In some embodiments, a pharmaceutical composition of the invention is administered at a dose of up to about 10 mg/kg. In some embodiments, a pharmaceutical composition of the invention is administered at a dose of about 0.002 mg/kg to about 7.5 mg/kg. In some embodiments, a pharmaceutical composition of the invention is administered at a dose of about 0.02 mg/kg to about 7.5 mg/kg. In some embodiments, a pharmaceutical composition of the invention is administered at a dose of about 0.05 mg/kg to about 7.5 mg/kg. In some embodiments, a pharmaceutical composition of the invention is administered at a dose of about 0.15 mg/kg to about 7.5 mg/kg. In some embodiments, a pharmaceutical composition of the invention is administered at a dose of about 0.02-6 mg/kg, about 0.05-6 mg/kg, about 0.15-6 mg/kg, about 0.3-6 mg/kg, about 0.6-6 mg/kg, about 1.3-6 mg/kg, or about 2.6-6 mg/kg. In some embodiments, a pharmaceutical composition of the invention is administered at a dose of about 0.002 mg/kg, about 0.006 mg/kg, about 0.02 mg/kg, about 0.05 mg/kg, about 0.15 mg/kg, about 0.3 mg/kg, about 0.6 mg/kg, about 1.3 mg/kg, about 2.0 mg/kg, about 2.6 mg/kg, about 3.0 mg/kg, about 3.5 mg/kg, about 3.9 mg/kg, about 4.25 mg/kg, about 5.0 mg/kg, about 5.75 mg/kg, about 6.0 mg/kg, about 6.5 mg/kg, about 7.0 mg/kg, or about 7.5 mg/kg.

In some embodiments, a method of the invention is for treating a patient having locally advanced or metastatic disease that is refractory to standard therapy.

In some embodiments, a method of the invention is for treating a patient having a histologically or cytologically confirmed malignant solid tumor associated with Nectin-4 expression, including, for example, urothelial (transitional cell) carcinoma; head and neck squamous cell carcinoma; non-small cell lung cancer; ovarian cancer; breast cancer; gastric cancer; or esophageal carcinoma. In some embodiments, a method of the invention is for treating a urothelial (transitional cell) carcinoma patient. In some embodiments, a method of the invention is for treating a head and neck squamous cell carcinoma patient. In some embodiments, a method of the invention is for treating a non-small cell lung cancer patient. In some embodiments, a method of the invention is for treating an ovarian cancer patient. In some embodiments, a method of the invention is for treating a breast cancer patient. In some embodiments, a method of the invention is for treating a gastric cancer patient. In some embodiments, a method of the invention is for treating an esophageal carcinoma patient.

In some embodiments, a method of the invention is for treating a patient having radiographically documented metastatic or locally advanced disease per Response Evaluation Criteria in Solid Tumors (RECIST) 1.1.

In some embodiments, a method of the invention is for treating a patient with non-measurable disease. In some embodiments, a method of the invention is for treating a patient with measurable disease.

In some embodiments, a method of the invention is for treating a patient at least 18 years of age.

In some embodiments, a method of the invention is for treating a patient having an Eastern Cooperative Oncology Group (ECOG) performance status score of 0 or 1 (Score 0: Fully active, able to carry on all pre-disease performance without restriction; Score 1: Restricted in physically strenuous activity but ambulatory and able to carry out work of a light or sedentary nature, e.g., light housework, office work).

In some embodiments, a patient has acceptable organ function, and one or more of the following condition:
Renal function with creatinine clearance (CLcr)≥60 mL/min by the Cockcroft-Gault equation or local equivalent;
Total bilirubin ≤1.5×upper limit of normal (ULN);
Aspartate aminotransferase (AST)≤2.5×ULN or ≤5×ULN in the presence of liver metastases;
Alanine aminotransferase (ALT)≤2.5×ULN or ≤5×ULN in the presence of liver metastases; and
International normalized ratio <1.5 or ≤institutional ULN.

In some embodiments, a method of the invention is for treating a patient with renal impairment having a CLcr>30 to 59 mL/min by the Modification of Diet in Renal Disease (MDRD) equation at screening.

In some embodiments, a patient has acceptable hematologic function, and one or more of the following condition:
Hemoglobin ≥9 g/dL;
Absolute neutrophil count (ANC)≥1500 cells/mm³; and
Platelet count ≥75,000 cells/mm³.

In some embodiments, a method of the invention is for treating a female patient, who is not lactating or pregnant as documented by a negative serum pregnancy (beta-human chorionic gonadotropin) test at screening and a negative urine pregnancy test within 72 hours before the first dose of a pharmaceutical composition of the invention.

In some embodiments, a method of the invention is for treating a patient, who is not or has not been administered any cytotoxic, small molecule, or other systemic chemotherapy within 14 days prior to the first dose of a pharmaceutical composition of the invention.

In some embodiments, a method of the invention is for treating a patient, who is not or has not been administered any immunotherapy, including monoclonal antibodies, within 28 days or 5 half-lives of the first dose of a pharmaceutical composition of the invention.

In some embodiments, a method of the invention is for treating a patient, who is not or has not been administered any cell-based therapy, including chimeric antigen receptor T cell therapy, within 60 days of the first dose of a pharmaceutical composition of the invention.

In some embodiments, a method of the invention is for treating a patient, who is not or has not been administered any experimental treatment(s), other than systemic anticancer therapies, within 28 days or 5 half-lives of the first dose of a pharmaceutical composition of the invention.

In some embodiments, a method of the invention is for treating a patient, who is not or has not been administered any radiation therapy within 28 days of the first dose of a pharmaceutical composition of the invention.

In some embodiments, a method of the invention is for treating a patient, who has not had an invasive surgery, excluding placement of vascular access, within 28 days of the first dose of a pharmaceutical composition of the invention. In some embodiments, a method of the invention is for treating a patient, who has not had a minimally invasive (laparoscopic, interventional radiology, or robotic) surgery within 14 days of the first dose of a pharmaceutical composition of the invention.

In some embodiments, a method of the invention is for treating a patient, who is not having or has not been administered any CD137 targeted therapy prior to the first dose of a pharmaceutical composition of the invention.

In some embodiments, a method of the invention is for treating a patient, who has not had any red blood cell transfusions, platelet transfusions, or growth factors within 14 days of the first dose of a pharmaceutical composition of the invention.

In some embodiments, a method of the invention is for treating a patient with renal impairment, who has received erythropoietic growth factors within 4 weeks of the first dose of a pharmaceutical composition of the invention.

In some embodiments, a patient does not have prior treatment-related toxicities that have not resolved to Grade 2 per National Cancer Institute (NCI) Common Terminology Criteria for Adverse Events (CTCAE) version 5.0 prior to the first dose of a pharmaceutical composition of the invention.

In some embodiments, a method of the invention is for treating a patient who has a body mass index <35 kg/m².

In some embodiments, a patient does not have a significant medical condition such as conditions affecting the skin including autoimmune conditions, for example, moderate/severe and active eczema or psoriasis. In some embodiments, a patient does not have an active uncontrolled systemic infection. In some embodiments, a patient does not have organ system dysfunction, including, for example, severe ascites, coagulopathy, or encephalopathy. In some embodiments, a patient does not have any gastrointestinal, skin, or pulmonary co-morbidities.

In some embodiments, a patient does not have a history of a cerebral vascular event, including, for example, stroke or transient ischemic attack, unstable angina, myocardial infarction, or signs or symptoms of New York Heart Association Class III to IV heart failure documented within 6 months prior to the first dose of a pharmaceutical composition of the invention.

In some embodiments, a patient does not have mean resting QTc (e.g., QTcF)>470 msec on triplicate electrocardiograms (ECGs).

In some embodiments, a patient does not have any factors that increase the risk of QTc prolongation or the risk of arrhythmic events, including, for example, severe heart failure, hypokalemia, congenital long QT syndrome, family history of long QT syndrome, unexplained sudden death under 40 years of age, or any concomitant medication use known to prolong the QT interval and/or cause torsades de pointes.

In some embodiments, a patient does not have any clinically important abnormalities in rhythm, conduction, or morphology of resting ECGs, including, for example, such as complete left bundle branch block or third-degree heart block not controlled by pacemaker or otherwise.

In some embodiments, a patient does not have uncontrolled diabetes with glycosylated hemoglobin ≥8%.

In some embodiments, a patient does not have uncontrolled symptomatic brain metastases.

In some embodiments, a method of the invention is for treating a patient with treated brain metastases, wherein the patient has had no progression for at least 4 weeks after central nervous system-directed treatment.

In some embodiments, a patient does not have uncontrolled hypertension (with repeated measurement of systolic blood pressure [BP]≥160 mmHg or diastolic BP≥100 mmHg that is not responsive to intervention).

In some embodiments, a patient does not have an HIV infection or acquired immune deficiency syndrome. In some embodiments, a patient with HIV infection is permitted if the patient meets all the following criteria at inclusion: a. CD4+ T-cell (CD4+) counts ≥350 cells/uL; b. on established antiretroviral therapy (ART) for at least 4 weeks; and c. HIV viral load <400 copies/mL.

In some embodiments, a patient does not have an active hepatitis B virus (HBV) infection.

In some embodiments, a patient has a controlled (treated) hepatitis B virus (HBV) infection, wherein an anti-viral therapy for HBV is administered at least 1 month prior to the first dose of a pharmaceutical composition of the invention.

In some embodiments, a patient has a controlled (treated) hepatitis B virus (HBV) infection, wherein HBV viral load is <2000 IU/mL (104 copies/mL) prior to the first dose of a pharmaceutical composition of the invention.

In some embodiments, a patient has a controlled (treated) hepatitis B virus (HBV) infection with viral load <2000 IU/mL (104 copies/mL), and continues receiving the same anti-viral HBV therapy throughout study treatment.

In some embodiments, a patient does not have an active hepatitis C virus (HCV) infection.

In some embodiments, a patient has received HCV treatment and has a sustained virologic response at 12 weeks (SVR12) or 24 weeks (SVR24) for 4 weeks prior to the first dose of a pharmaceutical composition of the invention.

In some embodiments, a patient does not have any prior or concurrent malignancy within 3 years prior to the first dose of a pharmaceutical composition of the invention. In some embodiments, a patient does not have any evidence of residual disease from a previously diagnosed malignancy prior to the first dose of a pharmaceutical composition of the invention.

In some embodiments, a patient has adequately managed a prior or concurrent malignancy with curative-intent treatment for basal cell carcinoma, squamous cell carcinoma of the skin, cervical intraepithelial neoplasia, cervical carcinoma in situ, melanoma in situ, or ductal carcinoma in situ of the breast.

In some embodiments, a patient has not received any live or attenuated vaccine within 30 days of the first dose of a pharmaceutical composition of the invention.

In some embodiments, a patient is not diagnosed with or does not have a clinically relevant immunodeficiency.

In some embodiments, a patient is not on use of prednisone ≥10 mg daily or equivalent or another strong systemic immunosuppressant, including, for example, calcineurin inhibitors, anti-proliferative agents, mTOR [mammalian target of rapamycin] inhibitors.

In some embodiments, a patient has not received intravenous anti-infective treatment within 14 days prior to the first dose of a pharmaceutical composition of the invention.

In some embodiments, a patient does not have a fever not attributable to the underlying illness within 14 days prior to the first dose of a pharmaceutical composition of the invention.

In some embodiments, a patient has not had any Prior organ or hematopoietic cell transplant, including allogeneic hematopoietic cell transplant.

In some embodiments, a patient does not have an autoimmune disease.

In some embodiments, a patient has well-controlled diabetes mellitus, alopecia, well-controlled thyroid disease, or vitiligo.

In some embodiments, a patient does not have an interstitial lung disease.

### Exemplary Cancers

In another aspect, the invention provides a method for treating cancer in a patient comprising administering to the patient a pharmaceutical composition as described herein. In some embodiments, the invention provides a method for treating cancer in a patient comprising administering to the patient by IV infusion an aqueous pharmaceutical composition comprising BT7480, or a pharmaceutically acceptable salt thereof, Tris base, mannitol, sodium hydroxide, sodium chloride, and water.

In some embodiments, the cancer is associated with Nectin-4. In some embodiments, the cancer is high Nectin-4 expressing.

In some embodiments, the cancer is a solid tumor.

In some embodiments, the cancer is bladder cancer. In some embodiments, the bladder cancer is selected from the group consisting of basal, p53-like, and luminal.

In some embodiments, the cancer is endometrial cancer. In some embodiments, the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated.

In some embodiments, the cancer is esophageal cancer. In some embodiments, the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI).

In some embodiments, the cancer is glioblastoma. In some embodiments, the glioblastoma is selected from the group consisting of proneural, neural, classical, and mesenchymal.

In some embodiments, the cancer is mesothelioma. In some embodiments, the mesothelioma is selected from the group consisting of pleural mesothelioma, peritoneal mesothelioma, pericardial mesothelioma, epithelioid mesothelioma, sarcomatoid mesothelioma, biphasic mesothelioma, and malignant mesothelioma.

In some embodiments, the cancer is multiple myeloma. In some embodiments, the multiple myeloma is selected from the group consisting of hyperdiploid, non-hyperdiploid, cyclin D translocation, MMSET translocation, MAF translocation, and unclassified.

In some embodiments, the cancer is ovarian cancer. In some embodiments, the ovarian cancer is selected from the group consisting of clear cell, endometrioid, mucinous, high-grade serous and low-grade serous ovarian cancer.

In some embodiments, the cancer is pancreatic cancer. In some embodiments, the pancreatic cancer is selected from the group consisting of squamous, pancreatic progenitor, immunogenic, and ADEX (Aberrantly Differentiated Endocrine eXocrine) pancreatic cancer.

In some embodiments, the cancer is prostate cancer. In some embodiments, the prostate cancer is selected from the group consisting of AZGP1 (subtype I), MUC1 (subtype II), and MUC1 (subtype III) prostate cancer.

In some embodiments, a cancer is a lung cancer. In some embodiments, a lung cancer is a met-amplified squamous NSCLC, a squamous cell NSCLC with wild type EGFR, or a T790M EGFR-expressing lung adenocarcinoma.

In some embodiments, a cancer is a breast cancer. In some embodiments, a breast cancer is a triple negative breast cancer. In some embodiments, a breast cancer is a basaloid triple negative breast cancer.

In some embodiments, a cancer is a colon cancer. In some embodiments, a cancer is a colorectal adenocarcinoma. In some embodiments, a colorectal adenocarcinoma is a high pgp-expressing colorectal adenocarcinoma.

In some embodiments, a cancer is a gastric cancer. In some embodiments, a gastric cancer is a FGFR-amplified gastric cancer.

In some embodiments, a cancer is a head and neck cancer. In some embodiments, a head and neck cancer is a nasal septum squamous cell carcinoma.

In some embodiments, a cancer is a sarcoma. In some embodiments, a sarcoma is a fibrosarcoma. In some embodiments, a fibrosarcoma is an N-ras mutant/IDH1 mutant soft tissue sarcoma (STS).

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC).

### EXEMPLIFICATION

### Example 1. Phase 1/2 Study of the Safety, Pharmacokinetics, and Preliminary Clinical Activity of BT7480 in Patients With Nectin-4 Associated Advanced Malignancies Primary Objectives

### Phase 1

To assess the safety and tolerability of BT7480, as a monotherapy and in combination with nivolumab, in patients with advanced solid tumors associated with Nectin-4 expression; and
To assess the safety and tolerability of BT7480 as a monotherapy in patients with renal insufficiency and advanced solid tumors.

### Phase 2

To assess the clinical activity of BT7480, as a monotherapy and in combination with nivolumab, in patients with advanced solid tumors associated with Nectin-4 expression.

### Secondary Objectives

### Phase 1

To assess the clinical activity of BT7480, as a monotherapy and in combination with nivolumab, in patients with advanced solid tumors associated with Nectin-4 expression; and
To assess the clinical activity of BT7480 as a monotherapy in patients with renal insufficiency and advanced solid tumors.

### Phase 2

To assess the safety and tolerability of BT7480, as a monotherapy and in combination with nivolumab, in patients with advanced solid tumors associated with Nectin-4 expression.

### Phase 1 and Phase 2

To assess additional measures of antitumor efficacy of BT7480 as a monotherapy and in combination with nivolumab;
To assess the pharmacokinetic (PK) parameters of BT7480, as a monotherapy and in combination with nivolumab, in patients with advanced solid tumors associated with Nectin-4 expression;
To assess the PK parameters of BT7480 as a monotherapy in patients with renal insufficiency and advanced solid tumors;
To evaluate the development of anti-drug antibodies (ADAs) in patients treated with BT7480; and
To evaluate cluster of differentiation (CD)137 target engagement in the peripheral blood of patients treated with BT7480 as a monotherapy and in combination with nivolumab.

### Exploratory Objectives

### Phase 1 and Phase 2

To evaluate potential pharmacodynamic (transcriptomic and proteomic) activity associated with PK/target engagement in blood and tumor of patients treated with BT7480;
To evaluate biomarkers associated with pharmacological activity in patients treated with BT7480;
To evaluate the pharmacogenomics (PGx) in patients treated with BT7480; and
To evaluate circulating tumor deoxyribonucleic acid (ctDNA) in patients treated with BT7480.

### Inclusion Criteria

Patients who meet all the following criteria will be eligible to participate in the study:
1. Must have locally advanced or metastatic disease that is refractory to standard therapy, or for which no standard therapy is judged to be appropriate or provide clinical benefit, as judged by the Investigator;
2. Must have a histologically or cytologically confirmed malignant solid tumor associated with Nectin-4 expression, including urothelial (transitional cell) carcinoma; head and neck squamous cell carcinoma; non-small cell lung cancer; and ovarian, breast, gastric, or esophageal carcinoma;
Note: Renal cell carcinoma and glioblastoma multiforme appear to have little to no Nectin-4 expression.
3. Must have radiographically documented metastatic or locally advanced disease per Response Evaluation Criteria in Solid Tumors (RECIST) 1.1;
Note: For monotherapy dose escalation, patients with non-measurable disease may also be enrolled into Cohorts 1 through 3, and patients with measurable disease only will be enrolled into Cohort 4 and beyond.
4. Must submit fresh or archival tumor tissue;
5. Must provide written informed consent, according to local guidelines, signed and dated by the patient or by a legal guardian prior to the performance of any study-specific procedures, sampling, or analysis;
6. Must be at least 18 years of age;
7. Must have an Eastern Cooperative Oncology Group (ECOG) performance status score of 0 or 1 (Score 0: Fully active, able to carry on all pre-disease performance without restriction; Score 1: Restricted in physically strenuous activity but ambulatory and able to carry out work of a light or sedentary nature, e.g., light housework, office work);
8. Must have acceptable organ function, as evidenced by all the following laboratory data:
a. Renal function with creatinine clearance (CLcr) ≥60 mL/min by the Cockcroft-Gault equation or local equivalent;
Note: Patients enrolling into the optional renal impairment cohorts should have a CLcr>30 to 59 mL/min by the Modification of Diet in Renal Disease (MDRD) equation at screening.
b. Total bilirubin ≤1.5× upper limit of normal (ULN);
Note: Patients with Gilbert's syndrome are eligible if their direct bilirubin is ≤1.5×ULN.
c. Aspartate aminotransferase (AST)≤2.5×ULN or ≤5×ULN in the presence of liver metastases;
d. Alanine aminotransferase (ALT)≤2.5×ULN or 5×ULN in the presence of liver metastases; and
e. International normalized ratio <1.5 or ≤institutional ULN.
9. Must have acceptable hematologic function, as evidenced by all the following laboratory data:
f. Hemoglobin ≥9 g/dL;
g. Absolute neutrophil count (ANC)≥1500 cells/mm³; and
h. Platelet count ≥75,000 cells/mm³.
10. Female patients must not be lactating or pregnant as documented by a negative serum pregnancy (beta-human chorionic gonadotropin) test at screening and a negative urine pregnancy test within 72 hours before the first dose of study drug. All females will be considered to be of childbearing potential unless they are postmenopausal (with amenorrhea for at least 12 months and follicle-stimulating hormone in the postmenopausal range at screening) or surgically sterile;
11. Fertile male patients and female patients of childbearing potential who are at risk of pregnancy must agree to use a highly effective (less than 1% failure rate) protocol-recommended method of birth control in conjunction with an acceptable but not highly effective method (more than 1% failure rate) during their participation in the study, and until 3 months following the last dose of BT7480 and, if applicable, until 5 months following the last dose of nivolumab;
12. Fertile male patients must agree to refrain from sperm donation from Day 1 until at least 3 months following the last dose of BT7480 and, if applicable, until 5 months following the last dose of nivolumab;
13. Must have life expectancy ≥12 weeks after the start of study drug per Investigator's judgment; and
14. Must be willing and able to comply with the protocol, the scheduled visits, treatment plan, study restrictions, laboratory tests, contraceptive guidelines, and other study procedures.

### Exclusion Criteria

Patients who meet any of the following criteria will be excluded from participation in the study:
1. Prior therapy with any of the following:
i. Cytotoxic, small molecule, or other systemic chemotherapy within 14 days of the first dose of study drug;
j. Immunotherapy, including monoclonal antibodies, within 28 days or 5 half-lives of the first dose of study drug, whichever is shorter;
k. Cell-based therapy, including chimeric antigen receptor T cell therapy, within 60 days of the first dose of study drug;
l. Experimental treatment(s), other than systemic anticancer therapies, within 28 days or 5 half-lives of the first dose of study drug, whichever is shorter;
m. Radiation therapy within 28 days of the first dose of study drug;
n. Major surgery, excluding placement of vascular access, within 28 days of the first dose of study drug for invasive surgery or 14 days of the first dose of study drug for minimally invasive (laparoscopic, interventional radiology, or robotic) surgical procedures;
Note: Patients must have recovered adequately after surgery and prior to starting study drug.
o. CD137 targeted therapy; or
p. Red blood cell transfusions, platelet transfusions, or growth factors within 14 days of the first dose of BT7480.
Note: Patients in the optional renal impairment cohorts may receive erythropoietic growth factors within 4 weeks of the first dose of BT7480, if clinically indicated.
2. Prior treatment-related toxicities that have not resolved to Grade 2 per National Cancer Institute (NCI) Common Terminology Criteria for Adverse Events (CTCAE) version 5.0;
3. Body mass index ≥35 kg/m²;
4. Known sensitivity to any of the ingredients of the study drug(s);
5. Significant medical condition which, in the Investigator's opinion, could compromise or interfere with the patient's safety or integrity of the study outcomes, such as conditions affecting the skin (conditions related to, or that may confound monitoring for rash, including autoimmune conditions such as moderate/severe and active eczema or psoriasis), immediate life-threatening illness (other than cancer), active uncontrolled systemic infection, organ system dysfunction (e.g., severe ascites, coagulopathy, or encephalopathy), or other gastrointestinal, skin, or pulmonary (after review of screening chest computed tomography [CT] if clinically indicated) co-morbidities;
6. History of a cerebral vascular event (e.g., stroke or transient ischemic attack), unstable angina, myocardial infarction, or signs or symptoms of New York Heart Association Class III to IV heart failure documented within 6 months prior to the first dose of study drug;
7. Mean resting QTc (e.g., QTcF) >470 msec on triplicate electrocardiograms (ECGs) obtained at screening;
8. Any factors that increase the risk of QTc prolongation or the risk of arrhythmic events such as severe heart failure, hypokalemia, congenital long QT syndrome, family history of long QT syndrome, unexplained sudden death under 40 years of age, or any concomitant medication use known to prolong the QT interval and/or cause torsades de pointes;
9. Any clinically important abnormalities, as assessed by the Investigator, in rhythm, conduction, or morphology of resting ECGs, such as complete left bundle branch block or third-degree heart block not controlled by pacemaker or otherwise;
10. Uncontrolled diabetes with glycosylated hemoglobin ≥8%;
11. Uncontrolled symptomatic brain metastases;
Note: Patients with treated brain metastases are eligible if there is no evidence of progression for at least 4 weeks after central nervous system-directed treatment
12. Uncontrolled hypertension (with repeated measurement of systolic blood pressure [BP]≥160 mmHg or diastolic BP≥100 mmHg that is not responsive to intervention) at screening or prior to initiation of study drug;
13. Known HIV infection or acquired immune deficiency syndrome;
Note: Well controlled HIV will be allowed if the patient meets all the following criteria at inclusion:

$$a . CD 4 + T - cell ( CD 4 + ) counts \geq 350 cells / uL ;$$

b. On established antiretroviral therapy (ART) for at least 4 weeks
c. HIV viral load <400 copies/mL
14. Active hepatitis B virus (HBV) infection;
Note: Controlled (treated) hepatitis will be allowed if the patient meets all the following criteria:
q. Anti-viral therapy for HBV must be given for at least 1 month prior to the first dose of study drug;
r. HBV viral load must be <2000 IU/mL (104 copies/mL) prior to the first dose of study drug; and
s. Those on active HBV therapy with viral load <2000 IU/mL (104 copies/mL) should stay on the same anti-viral therapy throughout study treatment.
15. Active hepatitis C virus (HCV) infection;
Note: Successfully treated patients with chronic HCV infection, defined as sustained virologic response at 12 weeks (SVR12) or 24 weeks (SVR24), are allowed if there is a 4-week period between achieving sustained viral response (SVR12 or SVR24) and starting the study drug.
16. Prior or concurrent malignancy whose natural history or treatment has the potential to interfere with the safety or efficacy assessment of the investigational regimen, particularly those patients with a history of another malignancy within 3 years prior to the first dose of study drug or any evidence of residual disease from a previously diagnosed malignancy, excluding adequately managed with curative-intent treatment for basal cell carcinoma, squamous cell carcinoma of the skin, cervical intraepithelial neoplasia, cervical carcinoma in situ, melanoma in situ, or ductal carcinoma in situ of the breast
17. Receipt of live or attenuated vaccine within 30 days of first dose of study drug;
Note: Patients who have received vaccination for Coronavirus Disease 2019 will be considered on a case-by-case basis after discussion with the Medical Monitor.
18. Diagnosis of a clinically relevant immunodeficiency;
19. Use of prednisone ≥10 mg daily or equivalent or another strong systemic immunosuppressant (e.g., calcineurin inhibitors, anti-proliferative agents, mTOR [mammalian target of rapamycin] inhibitors);
20. Receipt of intravenous anti-infective treatment or presence of fever not attributable to the underlying illness within 14 days prior to the first dose of study drug;
21. Psychological, familial, sociological, or geographical conditions that do not permit compliance with the protocol and/or follow-up procedures outlined in the protocol;
22. Any clinically significant safety concern related to prior checkpoint inhibitor(s) therapy;
23. Prior organ or hematopoietic cell transplant, including allogeneic hematopoietic cell transplant;
24. History of autoimmune disease, except well-controlled diabetes mellitus, alopecia, well-controlled thyroid disease, or vitiligo;
25. History of interstitial lung disease; or
26. Prior or current evidence of any condition, therapy, or laboratory abnormality that might confound the results of the study, interfere with the patient's participation or, in the opinion of the Investigator, is considered to not be in the best interest of the patient to participate in the study.

### Study Design and Duration

This is a Phase 1/2 first-in-human (FIH), open-label, dose escalation, dose confirmation, and dose expansion multicenter study to evaluate the safety, PK, and clinical activity of BT7480 in patients with advanced solid tumors associated with Nectin-4 expression. Dose escalation and dose expansion of BT7480 monotherapy, with a possible renal impairment cohort will be the focus of the Phase 1 portion of the study. A subsequent dose escalation and dose expansion cohort evaluating BT7480 in combination with nivolumab may also be evaluated as part of Phase 1. Enrollment into optional renal impairment cohorts may be concurrent with enrollment to monotherapy dose expansion/combination therapy dose escalation cohorts. Phase 2 of the study plans to evaluate the efficacy of BT7480 as a monotherapy or in combination with nivolumab dosed at the recommended Phase 2 dose (RP2D) determined in Phase 1. The study is planned to be conducted in up to 20 clinical sites globally.

The study will enroll patients with histologically or cytologically confirmed malignant solid tumors associated with Nectin-4 expression. Patients must have locally advanced or metastatic disease that is refractory to standard therapy, or for which no standard therapy is judged to be appropriate or provide clinical benefit, as judged by the Investigator.

Patients must submit fresh or archival tumor tissue at baseline, have an ECOG performance status score 0 or 1, have protocol-defined acceptable organ and hematologic function, and have a life expectancy ≥12 weeks. Tumor associated Nectin-4 expression levels will be determined retrospectively for all patients using archival tumor tissue or fresh tumor biopsy collected during screening.

During Phase 1 monotherapy (dose escalation [Cohort 3 onwards], expansion, and renal impairment cohorts) and Phase 1 combination therapy (dose escalation and expansion cohorts), patients may provide optional paired (pre- and on-treatment) tumor biopsies. If the patient has consented to optional paired tumor biopsies, both a pre-treatment baseline tumor biopsy and archival tumor tissue are requested. On-treatment biopsy sampling is allowed at any time after Cycle 1 Day 1 and, if applicable and possible, at relapse.

Safety and tolerability will be assessed from the time of informed consent until the safety follow-up visit by vital signs, physical examinations, 12-lead ECGs, routine clinical laboratory evaluations (chemistry, hematology, coagulation, and urinalysis), ADAs, and adverse events (AEs). For cohorts that receive combination therapy with nivolumab, additional monitoring for autoimmune and endocrine disorders is scheduled.

For disease assessment by Investigator per RECIST 1.1, a CT or magnetic resonance imaging scan, or manual measurement of visual lesions or other types of imaging, should be performed at screening; every 8 weeks (±7 days) for the first 12 months; and every 12 weeks (128 days) thereafter until disease progression, death, or another response withdrawal criterion is met. Starting with Cycle 3, disease assessment by Investigator per RECIST 1.1 should be performed at the start of every odd cycle (±7 days) through 12 months, then every 12 weeks (128 days) thereafter until disease progression, death, or another response withdrawal criterion is met. It is also acknowledged that disease progression may require, in certain circumstances, further evaluation for possible pseudo-progression.

PK of BT7480 will be assessed using serial blood and urine samples obtained at prespecified time points. Additionally, tumor concentrations will be analyzed in a similar manner, if available. Pharmacodynamic response to treatment will be assessed by biomarker analyses (e.g., CD137 target engagement [circulating mechanistic proteins and flow cytometry-based receptor occupancy], exploratory transcriptomic and proteomic immune profiling in blood and tumor [inflammatory cytokines, neutralizing antibodies, flow cytometry immunophenotyping, multiplex immunofluorescence, RNA immune profiling]). A single blood sample for germline genomics will be collected pre-dose on Cycle 1 Day 1 and blood samples for ctDNA PGx will be collected at prespecified timepoints.

The study duration will be approximately 36 months: approximately 24 months for dose escalation and approximately 12 months for dose expansion.

Patients who meet eligibility criteria during screening (Week −4 up to Week 0) will be enrolled into the study.

### Phase 1 Dose Escalation—BT7480 Monotherapy

The starting dose for the BT7480 monotherapy dose escalation cohort is planned to be 0.002 mg/kg. Patients will be assigned sequentially to increasing BT7480 doses (see Table 1). BT7480 dose will be escalated in subsequent cohorts after patient(s) enrolled in each cohort have completed both the planned doses as well as the dose-limiting toxicity (DLT) period (28 days post-first dose). Patients who miss a planned dose within the DLT period but experience a DLT will be considered DLT evaluable. The monitoring for liver dysfunction will continue for the entire duration of a patient's participation in the study.

Patients with non-measurable disease may also be enrolled into Cohorts 1 through 3, and patients with measurable disease only will be enrolled into Cohort 4 through 13. At least 1 evaluable patient will be enrolled in each of the first 4 cohorts unless a subject in one of these cohorts experiences a Grade 2 adverse event or higher that was at least possibly related to study drug. Should that occur, the next subsequent cohort will contain at least 3 evaluable patients; further expansion with 3 additional subjects is required if a subject experience a DLT. If a subject experiences a grade 3 or higher toxicity in any of the single patient expansion cohorts then the current cohort will be expanded to three patients at least.

TABLE 1
Dose Plan: Monotherapy Dose Escalation Cohorts

| Dose Cohort | BT7480 Dose Level^{a} | Number of Patients |
|---|---|---|
| Cohort 1 | 0.002 mg/kg | 1 |
| (starting dose) | | |
| Cohort 2 | Not to exceed 0.006 mg/kg | 1 to 3 |
| Cohort 3 | Not to exceed 0.02 mg/kg | 1 to 3 |
| Cohort 4 | Not to exceed 0.05 mg/kg | 1 to 3 |
| Cohort 5 | Not to exceed 0.15 mg/kg | 3 to 6 |
| Cohort 6 | Not to exceed 0.3 mg/kg | 3 to 6 |
| Cohort 7 | Not to exceed 0.6 mg/kg | 3 to 6 |
| Cohort 8 | Not to exceed 1.3 mg/kg | 3 to 6 |
| Cohort 8A^{b} | Not to exceed 2.0 mg/kg | 3 to 6 |
| Cohort 9 | Not to exceed 2.6 mg/kg | 3 to 6 |
| Cohort 9A^{b} | Not to exceed 3.0 mg/kg | 3 to 6 |
| Cohort 10 | Not to exceed 3.5 mg/kg | 3 to 6 |
| Cohort 10A^{b} | Not to exceed 4.25 mg/kg | 3 to 6 |
| Cohort 11 | Not to exceed 5.0 mg/kg | 3 to 6 |
| Cohort 11A^{b} | Not to exceed 5.75 mg/kg | 3 to 6 |
| Cohort 12 | Not to exceed 6.5 mg/kg | 3 to 6 |
| Cohort 12A^{b} | Not to exceed 7.0 mg/kg | 3 to 6 |
| Cohort 13 | Not to exceed 7.5 mg/kg | 3 to 6 |

^{a}BT7480 will be administered as an intravenous infusion over 60 (−5/+15) min, QW.
^{b}Cohorts with an A suffix correspond to intermediate dose levels that may be explored based on SRC recommendation.
min = minute(s); SRC = Safety Review Committee; QW = once weekly.

Subsequent dose escalation cohorts (Cohort 5 onwards or an earlier cohort if a 3 patient expansion of that cohort has been triggered) will enroll patients based on a 3+3 design, wherein 3 patients will be initially enrolled and treated at each dose level. The 3+3 design and plan of action should a DLT occur are provided in Table 2. In all 3+3 cohorts, there will be at least 48 hours between the first patient dosing and subsequent patient dosing. Treatment cycles will occur consecutively, as per the Schedule of Assessments (SOA) in Table S1.

TABLE 2
3 + 3 Design: Monotherapy Dose Escalation Cohorts

| Number of Patients With a DLT | Action |
|---|---|
| 0 of 3 patients | Escalate to the next dose level |
| 1 of 3 patients | Accrue up to 3 additional evaluable |
| | patients at the current dose level |
| | (for a total of up to 6 evaluable |
| | patients) |
| 1 of 6 patients | Escalate to the next dose level |
| ≥2 patients in a dose level of | The MTD has been exceeded |
| up to 6 patients | |

DLT = dose-limiting toxicity; MTD = maximum tolerated dose.

In the 3+3 design, if 1 patient experiences a DLT, 3 additional patients will be treated with the same dose. An evaluation of a cohort of at least 3 patients completing the DLT period (28 days post-first dose) is required prior to proceeding to the next dose level. If ≥2 patients in a dose level of up to 6 patients experience DLTs, the maximum tolerated dose (MTD) has been exceeded. If 2 patients in a dose level of up to 6 patients experience DLTs and only 3 patients were evaluated at the previous (next lower) dose, then 3 additional patients will be evaluated at the previous (next lower) dose; and if 0 or 1 patient has a DLT, then the Safety Review Committee (SRC) will consider enrolling patients at the intermediate dose level below the proceeding dose level as specified in Table 1. If there are no DLTs at the intermediate dose level escalation, the SRC will meet to decide the MTD.

The MTD will be defined according to the above guidelines; however, if subsequent to the SRC decision, further data becomes available that might change the attribution of causality or severity of a given event (e.g., based on subsequent evolution of events, follow-up investigations, or follow-up observations), the SRC may decide to recommence with dose escalation and a previous MTD could be rescinded. The RP2D, not exceeding the MTD, will be determined by the SRC based on available safety, PK, and pharmacodynamic data, including efficacy data.

The window of observation for DLTs is designed to assess predominantly acute events. Longer term and possibly immune-related AEs with specific attention applied to liver function, which may accrue over several cycles of therapy, will be considered in the assessments undertaken during each SRC meeting.

Following completion of the DLT period for each cohort, the SRC will review the available safety, PK, and pharmacodynamic data, including efficacy data, to determine the potential for dose escalation and the recommended dose level for the next cohort. The data must meet the adequate minimum standards (as specified in the SRC Charter) such that the SRC is satisfied to continue with dosing.

Intra-patient dose escalation: At the discretion of the Medical Monitor and Investigator, patients in preceding lower dose cohorts may receive a higher dose one dose level below the highest dose level deemed safe by a formal dose escalation decision by the SRC. For example, if a patient is tolerating 0.006 mg/kg and the 0.05 mg/kg has been deemed safe, the patient may escalate to the 0.02 mg/kg dose. The patient's clinical experiences in the higher dose cohort will be considered as part of the totality of data by the SRC but will not contribute to formal dose escalation decisions that have already been made.

Optional backfill cohorts: During dose escalation, selected dose levels may be further expanded (also known as backfilling) to include up to 10 patients per given dose level in order to further evaluate tolerability, PK, pharmacodynamics, and biological activity of BT7480. To help ensure that patients are not unnecessarily enrolled to subtherapeutic dose levels, these dose level expansions will only start when a dose level already declared safe by the SRC also demonstrates evidence of therapeutic exposures, target engagement, or Investigator-assessed response.

Subsequent dose levels above this would also be eligible for expanded enrollment after being declared safe by the SRC. If slots are available in both the optional backfill cohort as well as the dose escalation cohort simultaneously, enrollment into the dose escalation cohort will take precedent.

Withdrawn patients or patients who end treatment prior to study completion for reasons other than a DLT and who do not meet the minimum requirements for inclusion in the MTD determining population will be replaced, where necessary, to ensure adequate safety assessment for each cohort.

### Phase 1 Dose Confirmation—Optional BT7480 Monotherapy Renal Impairment Cohorts

Subsequent to SRC determination of a BT7480 monotherapy dose, 2 cohorts of 6 patients each with moderate renal insufficiency (CLcr >30 to 59 mL/min by the MDRD equation or local equivalent) will be enrolled at a dose based on the totality of PK, pharmacodynamic, safety and efficacy in patients with normal renal function and mild renal impairment (creatinine clearance ≥60 mL/min). If there is sufficient concern around the therapeutic index during dose escalation, then the SRC will direct a lower dose for the renal cohort. Six patients with CLcr 50 to 59 mL/min will enroll first, followed by 6 patients with CLcr >30 to 49 mL/min. Treatment cycles will occur consecutively, as shown in Table S2.

If ≥2 patients in a renal impairment cohort of ≤6 patients experience a DLT during the DLT period (28 days post-first dose), the MTD has been exceeded and enrollment will be stopped pending SRC review. The SRC will review the available safety, PK, and pharmacodynamic data, including efficacy data, to determine if additional cohorts/patients are needed. Withdrawn patients or patients who end treatment prior to study completion for reasons other than a DLT and who do not meet the minimum requirements for inclusion in the MTD determining population will be replaced, where necessary, to ensure adequate safety assessment for each cohort.

### Phase 1 Dose Escalation—Optional Combination Therapy

Subsequent to SRC determination of a BT7480 monotherapy dose, a combination therapy (BT7480+nivolumab) dose escalation may be opened. The starting dose of BT7480 in combination therapy will be 1 dose level lower than the BT7480 monotherapy RP2D; see Table 3. Patients will enroll based on a 3+3 design, wherein 3 patients will be initially enrolled and treated at each dose level. Treatment cycles will occur consecutively, as shown in Table S3.

TABLE 3
Dose Levels: Combination Therapy Cohorts

| Dose Cohort | BT7480 Dose Level^{a} | Nivolumab Dose^{b} |
|---|---|---|
| Cohort −1 | Monotherapy dose^{c} - 2 dose levels | 240 mg |
| Cohort 1 | Monotherapy dose^{c} - 1 dose level | 240 mg |
| (starting dose) | | |
| Cohort 2 | Monotherapy dose^{c} | 240 mg |

^{a}Each dose of BT7480 will be administered as an intravenous infusion, QW. Based on SRC recommendations, reductions in dosing and visit schedule to every other week (Days 1 and 15) may be considered.
^{b}Each dose of nivolumab will be administered as an intravenous infusion, Q2W. The total duration of nivolumab administration will be based on patient response to treatment and Investigator judgment.
^{c}SRC-determined BT7480 monotherapy dose that has demonstrated safety, PK, pharmacodynamic (target engagement), and/or clinical activity.
PK = pharmacokinetic(s); SRC = Safety Review Committee; Q2W = once every 2 weeks; QW = once weekly.

If 1 of these 3 patients experiences a DLT during the DLT period (28 days post-first dose), the dose level will be expanded to 6 patients. BT7480 doses will be escalated in the subsequent cohorts after patients enrolled in each cohort have completed the planned doses of BT7480 and nivolumab as well as the DLT period. Patients who miss a planned dose within the DLT period but experience a DLT will be considered DLT evaluable.

If ≥2 patients experience a DLT in any cohort of ≤6 patients, the MTD has been exceeded. Following each cohort, the SRC will review available safety, PK, and pharmacodynamic data, including efficacy data, to determine if additional cohorts/patients are needed. The dose level will never exceed the BT7480 monotherapy RP2D.

If a patient has intercurrent events that are not thought to be related to BT7480, the patient may be considered evaluable for SRC consideration. If a patient has an event that is not a DLT, is related to BT7480, and is considered to be clinically significant by the SRC, that event will be evaluated for dose escalation determinations.

Withdrawn patients or patients who end treatment prior to study completion for reasons other than a DLT and who do not meet the minimum requirements for inclusion in the MTD determining population will be replaced, where necessary, to ensure adequate safety assessment for each cohort.

### Phase 1 Dose Expansion—Monotherapy and Optional Combination Therapy

Following the determination of the RP2D for either monotherapy (BT7480) or combination therapy (BT7480+nivolumab), the study will be expanded such that up to 20 patients in 2 cohorts will be exposed to the dose chosen to be taken forward for monotherapy and combination therapy. Treatment cycles will occur as shown in Table S1 for monotherapy and Table S3 for combination therapy.

### Phase 2 Efficacy Evaluation—Monotherapy and Optional Combination Therapy

Based upon the clinical activity observed in Phase 1 dose escalation and dose expansion, up to 2 cohorts of patients may enroll in 2 tumor-defined cohorts during Phase 2 of the study. BT7480 will be dosed at the RP2D determined from either the monotherapy or combination therapy cohorts in Phase 1. Treatment cycles will occur as shown in Table S1 for monotherapy and Table S3 for combination therapy.

### Dose-Limiting Toxicity

Toxicity will be assessed using the NCI CTCAE version 5.0, unless otherwise specified. A toxicity will be considered ‘dose limiting’, provided it meets the DLT definitions, if it occurs during the DLT period (28 days post-first dose), and is related to BT7480. Note that these criteria apply upon the first dose or combination received. The SRC will also take into account whether a supportive care agent is subsequently given. Ongoing measurement of relevant parameters, including ALT, AST, total bilirubin, and alkaline phosphatase (ALP), will extend beyond the DLT period and will be regularly examined by the SRC and individual Investigators alike with an eye on the history of CD137 agonistic targeted agents.

In combination therapy cohorts, toxicities known to be associated with nivolumab will not necessarily be determined to be DLTs unless they are unusual or of markedly increased severity or duration per Investigator determination. Patients in combination therapy cohorts with toxicities known to be directly associated with nivolumab that cause the withdrawal of nivolumab treatment may receive single agent BT7480 per Investigator discretion and remain on the study. Patients in combination therapy cohorts with toxicities assumed or known to be directly associated with BT7480 will also discontinue nivolumab and may remain on study for safety follow-up per Investigator discretion.

The DLT definitions include the following:

### 1. Hematologic AEs:

a. ≥Grade 4 neutropenia lasting >5 days;
b. Febrile neutropenia, defined as ANC <1000 cells/mm³ with a single temperature of 38.3° C. (101° F.) or a sustained temperature of 38° C. (100.4° F.) for >1 hour;
c. ≥Grade 4 thrombocytopenia;
d. Grade 3 thrombocytopenia associated with clinically significant bleeding; or
e. ≥Grade 3 anemia unexplained by underlying disease or pre-existing renal dysfunction (renal impairment cohort).

### 2. Non-Hematologic AEs:

a. ≥Grade 3 fatigue lasting ≥5 days;
b. Any ≥Grade 3 non-hematologic AE of any clinically significant duration occurring during the period from infusion start on Cycle 1 Day 1 through the end of the DLT period (except for ≥Grade 3 nausea, vomiting, or diarrhea that is responsive to supportive care and lasts ≤72 hours);
c. ≥Grade 3 hypertension;
d. Any related AE leading to the inability to deliver at least 75% of the planned total dose for the DLT period, except in situations other than toxicity (e.g., participant noncompliance, significant contribution of underlying disease condition, or logistical issues with drug delivery); or
e. Any elevation in aminotransferases and total bilirubin fulfilling drug-induced liver injury (Hy's law) criteria, that is, the simultaneous occurrence of the following components:
   ALT or AST>3×ULN (or >3×baseline for those with hepatic metastases);
   Total bilirubin >2×ULN (or >2×baseline for those with hepatic metastases);
   ALP<2×ULN; and
   No other reason can be found to explain the combination of increased aminotransferases and serum total bilirubin, such as viral hepatitis, alcohol abuse, ischemia, pre-existing liver disease, or another drug capable of causing the observed injury.

Events that will not automatically be considered a DLT include the following:
≤Grade 3 nausea, vomiting, or diarrhea lasting ≤72 hours;
≥Grade 3 electrolyte abnormality lasting ≤72 hours, is not clinically complicated, and resolves spontaneously or responds to conventional medical interventions;
≥Grade 3 amylase or lipase that is not associated with symptoms of clinical manifestations of pancreatitis; and
AEs clearly related to disease, pre-existing conditions, nivolumab, or environmental factors.

Patients who discontinue treatment early due to disease progression or withdrawal will be asked to have all end of treatment safety evaluations performed. Withdrawn patients or patients who end treatment prior to study completion for reasons other than a DLT and who do not meet the minimum requirements for inclusion in the MTD determining population will be replaced, where necessary, to ensure adequate safety assessment for each dose escalation and the optional renal impairment cohorts.

### Dosage Forms and Route of Administration

### BT7480

BT7480 will be provided in 4R injection vials containing 66 mg of BT7480 per vial. The formulation will be reconstituted with 1 mL of water for injections (resulting in a total reconstituted volume of 1.1 mL) to provide BT7480 at a target concentration of 60 mg/mL for further dilution into 0.9% saline infusion bags prior to intravenous administration (by infusion).

The dose will be calculated based upon dose cohort assignment and the patient's body weight in kg. Dose readjustments will not be performed during a cycle, unless there is a ≥10% change in body weight from Day 1 of the cycle.

Pre-medications (e.g., diphenhydramine, acetaminophen) are recommended at subsequent dosing events as needed, if manageable infusion-related reactions are observed. Steroids should be avoided as pre-medication/prophylaxis.

BT7480 will be administered as an intravenous infusion over 60 (−5/+15) minutes (i.e., infusion periods between 55 and 75 minutes are acceptable), QW.

### Nivolumab

Nivolumab will be provided as 40 mg/4 mL, 100 mg/10 mL, and 240 mg/24 mL vials. The dose administered during the study will be 240 mg Q2W.

Nivolumab should be diluted with either 0.9% sodium chloride injection or 5% dextrose injection to a final concentration ranging from 1 mg/mL to 10 mg/mL. The total volume of infusion must not exceed 160 mL.

Pre-medications for nivolumab should be per local standard of care (e.g., acetaminophen, diphenhydramine). Steroids should be avoided as pre-medication/prophylaxis.

Nivolumab 240 mg, as part of combination therapy, will be administered as an intravenous infusion over 30 (−5/+15) minutes Q2W. The total duration of nivolumab administration will be based on patient response to treatment and Investigator judgment.

On nivolumab-coincident dosing days, BT7480 should be administered first, followed by observation for at least an hour, and then nivolumab should be administered as an intravenous infusion after proper flushing of the initial line or via a separate line.

### Endpoints

### Primary Endpoints

### Phase 1

Incidence and severity of treatment-emergent adverse events (TEAEs), potentially including abnormalities in laboratory assessments, ECG findings, and vital signs using the NCI CTCAE version 5.0.

### Phase 2

Overall response rate (ORR), defined as complete response (CR) plus partial response (PR), by RECIST 1.1 based on Investigator assessment; and
Clinical benefit rate (CBR), defined as the proportion of patients with CR, PR, or stable disease (SD) for ≥8 weeks, by RECIST 1.1 based on Investigator assessment.

### Secondary Endpoints

### Phase 1

ORR by RECIST 1.1 based on Investigator assessment; and
CBR, defined as the proportion of patients with CR, PR, or SD for ≥8 weeks, by RECIST 1.1 based on Investigator assessment

### Phase 2

Incidence and severity of TEAEs, including abnormalities in laboratory assessments, ECG findings, and vital signs using the NCI CTCAE version 5.0.

### Phase 1 and Phase 2

Duration of response (DoR), defined as the time between the initial response to therapy (CR or PR) and subsequent disease progression, by RECIST 1.1;
Progression-free survival at 6 months and overall, time to progression, duration of response, overall survival at 12 months, and time to response in patients treated with BT7480 as a monotherapy or in combination with nivolumab;
PK parameters for BT7480 in patients with normal and decreased renal function treated with BT7480 as a monotherapy or in combination with nivolumab;
Incidence of ADAs in patients treated with BT7480 as a monotherapy or in combination with nivolumab; and
Determination of CD137 target engagement in peripheral blood of patients treated with BT7480 as a monotherapy or in combination with nivolumab.

### Exploratory Endpoints

### Phase 1 and Phase 2

Assessment of potential changes in immune cell activation (transcriptomic and proteomic profiling) in blood and tumor of patients treated with BT7480;
Assessment of potential changes in spatial proteomic profiles between immune cells and tumor cells in patients treated with BT7480;
Assessment of potential changes in soluble target and inflammatory cytokines in the plasma from baseline in patients treated with BT7480;
Assessment of biomarkers from baseline tumor and peripheral blood samples such as, but not limited to, Nectin-4, CD137, programmed cell death protein 1/PD-L1;
Evaluation of germline PGx in patients treated with BT7480; and
Quantification of ctDNA in patients treated with BT7480.

### Pharmacokinetic Assessments

PK characterization of BT7480, as a monotherapy and in combination with nivolumab, will be performed with validated bioanalytical method(s) and will include calculation of the following parameters using noncompartmental analysis:
Maximum plasma concentration (Cₘₐₓ);
Time to Cₘₐₓ;
Terminal half-life;
Area under the plasma concentration-time curve from time 0 to time t;
Area under the plasma concentration-time curve from time 0 to infinity;
Apparent total body clearance of the drug from plasma; and
Apparent volume of distribution.

The following PK parameters will be calculated using urine concentrations of BT7480 where available:
Cumulative amount of BT7480 excreted in the urine;
Renal clearance; and
Fraction of dose excreted renally.

### Correlative Testing:

Patients must submit fresh or archival tumor tissue at baseline for histologic or cytologic confirmation, for assessment of Nectin-4 expression levels, and additional molecular or genetic characterization such as, but not limited to, markers of immune status (e.g., programmed death-ligand 1 [PD-L1] expression and immune cell infiltration), proliferation (e.g., Ki-67), and/or immunogenic cell death.

Paired (pre- and on-treatment) tumor biopsies may be collected to investigate intratumoral PK and pharmacodynamic effects of BT7480.

Pre- and post-dose blood samples will be collected to assess pharmacodynamic response, biomarkers associated with BT7480 response, and treatment resistance biomarkers.

### Statistical Analyses:

The following analysis populations will be defined. Analysis populations will be defined separately for each study phase.

The Full Analysis Set will include all enrolled patients.

The Safety Analysis Set will include all patients who receive at least 1 dose of BT7480.

The Response-Evaluable Analysis Set will include all patients who receive at least 1 dose of BT7480 and have at least 1 evaluable post-baseline RECIST 1.1 assessment.

The PK Analysis Set will include all patients who receive at least 1 dose of BT7480 and have at least 3 measurable post-dose plasma concentrations.

Other analysis sets may be defined as appropriate and, if applicable, will be described in the Statistical Analysis Plan.

No formal hypothesis testing is planned, and summaries will in general be descriptive. Appropriate summaries will be provided for each assessment based on data type (continuous or categorical).

Unless described otherwise, analyses will in general be presented separately by study phase and BT7480 dose level (in Phase 1) and cohort (in Phase 2). As appropriate for Phase 1 summaries, renal impairment cohort patients and patients treated with combination therapy may be presented separately depending on the analysis.

For Phase 2, the primary efficacy analysis will present the point estimates of the ORR and CBR along with the associated 95% exact confidence intervals for the Safety Analysis Set. To be included in the calculation of ORR, the response should be confirmed at the next RECIST 1.1 assessment. If a patient has a response of SD (or better) occurring prior to 8 weeks and no subsequent assessment, then the patient will be counted as a failure in calculation of the CBR If the Safety Analysis Set and the Response-Evaluable Analysis Set differ, then a sensitivity analysis may be performed using the Response-Evaluable Analysis Set.

For secondary efficacy analysis, time-to-event endpoints (progression-free survival, time to progression, duration of response, overall survival, and time to response) will be summarized using Kaplan-Meier methods. Additional details regarding censoring rules will be provided in the Statistical Analysis Plan. The level of CD137 target engagement in peripheral blood will be summarized descriptively.

Safety analyses will be performed using the Safety Analysis Set. Safety assessments will be summarized descriptively with actual values and change from baseline for continuous variables. TEAEs, serious AEs, and DLTs will be summarized by System Organ Class and Preferred Term. Additional summaries of AEs by severity, seriousness, and relationship to study drug(s) will be presented. The incidence of ADAs will be summarized. Additional details will be provided in the Statistical Analysis Plan.

PK parameters for BT7480 will be calculated using standard noncompartmental analysis methods and summarized using descriptive statistics. PK concentration data will be summarized using descriptive statistics. Plasma BT7480 concentrations will be plotted against timepoints (linear and semi-logarithmic). Additional details will be provided in the Statistical Analysis Plan.

No formal interim analysis is planned in Phase 2.

Available safety, PK, and pharmacodynamic data, including efficacy data, will be reviewed on an ongoing basis by the SRC.

### Sample Size Determination

The entire study will enroll a total of approximately 200 patients.

### Phase 1

In Phase 1, the total number of patients enrolled will be approximately 110 patients (including optional cohorts). The sample size will be determined from the observed data and the RP2D determination. Approximately 40 patients are planned to be enrolled into monotherapy dose escalation cohorts. Approximately 12 patients with moderate renal impairment may be enrolled for dose confirmation at a dose based on the totality of PK, pharmacodynamic, safety and efficacy in patients with normal renal function and mild impairment (creatinine clearance 60≥mL/min). Following monotherapy RP2D determination, approximately 12 patients may be enrolled into combination therapy dose escalation cohorts.

Upon RP2D determination for monotherapy and combination therapy, approximately 20 additional evaluable patients may be enrolled into each monotherapy and combination therapy dose expansion cohort. Patients that do not have at least 1 post-baseline RECIST 1.1 assessment may be replaced in the dose expansion cohort.

An intended 12 patients for the moderate renal impairment cohorts are deemed sufficient to demonstrate any gross renal effects, for example, more than doubling of the area under the curve in the renally impaired. The degree of inter-individual variation is not currently known as this is a FIH study, and therefore, a formal sample size calculation cannot be undertaken.

In some indications such as bladder cancer, as many as 50% of patients might have some degree of renal impairment. This cohort allows for inclusion of such patients in early phase oncology research whilst also giving an initial gross estimate of the impact of renal impairment in this agent which is predicted to undergo significant renal filtration.

### Phase 2

After dose expansion, the protocol may proceed to include an additional 45 evaluable patients in each cohort for Phase 2. The total number of patients planned to be enrolled in Phase 2 is approximately 90 (including optional cohorts).

### Clinical Laboratory Analytes

Standard Safety Chemistry Panel

| | |
|---|---|
| Alanine aminotransferase | Albumin |
| Alkaline phosphatase | Amylase |
| Aspartate aminotransferase | Bicarbonate/carbon dioxide |
| Blood urea nitrogen | Calcium |
| Chloride | Creatine kinase |
| Creatinine | Creatinine clearance [1] |
| Glucose | Lactate dehydrogenase |
| Lipase | Potassium |
| Sodium | Total bilirubin |
| Total protein | Uric acid [2] |

1. Creatinine clearance in mL/min will be calculated using the Modification of Diet in Renal Disease equation for the optional renal impairment cohorts and using the Cockcroft-Gault equation or local equivalent for all other cohorts.
2. To be analyzed for optional renal impairment cohorts only.

Additional Chemistry Parameters

| | |
|---|---|
| Glycosylated hemoglobin | Magnesium |

Endocrinology

| | |
|---|---|
| Adrenocorticotropic hormone [1] | Follicle-stimulating hormone [2] |
| Human chorionic gonadotropin [3] | Thyroid hormones [1] [4] |

1. Only for patients receiving nivolumab.
2. Follicle-stimulating hormone in postmenopausal women with amenorrhea for at least 1 year at screening.
3. Serum or urine pregnancy test for women of childbearing potential only.
4. Includes free triiodothyronine, free thyroxine, and thyroid-stimulating hormone.

Hematology

| | |
|---|---|
| Hematocrit | Hemoglobin |
| Platelets | Red blood cell count |

### White Blood Cell Count and Differential [1]

1. Must include a 5-part white cell differential count.

Coagulation

| | |
|---|---|
| Fibrinogen | International normalized ratio |
| Prothrombin time | Partial thromboplastin time |

Immunology

| | |
|---|---|
| Antinuclear antibody [1] | Rheumatoid factor [1] |

1. Only for patients receiving nivolumab.

Urinalysis [1]

| | |
|---|---|
| Bilirubin | Blood |
| Glucose | Ketones |
| Leukocyte esterase | Microscopy |
| Nitrite | pH |
| Protein | Specific gravity |
| Urobilinogen | |

1. A full urinalysis must be performed at screening, Cycle 1 visits, and end of treatment, and may be substituted with a dipstick with reflex sedimentation at all other visits.

Viral Testing and Serology

| | |
|---|---|
| Hepatitis B core antibody | Hepatitis B DNA [1] |
| Hepatitis B surface antigen | Hepatitis C antibody |
| Hepatitis C RNA [2] | |

1. PCR for viral load is to be performed if positive for hepatitis B core antibody or hepatitis B surface antigen.
2. PCR for viral load is to be performed if positive for hepatis C antibody.
DNA=deoxyribonucleic acid; PCR=polymerase chain reaction; RNA=ribonucleic acid.

TABLE S1
BT7480 Monotherapy - Dose Escalation and Dose Expansion Cohorts - Phase 1 and Phase 2

| | Cycle 1^{a} | | | | | | | | | | | Safety | PFS | OS |
|---|---|---|---|---|---|---|---|---|---|---|---|---|---|---|
| Screen | (DLT Period = 28 Days)^{b} | | | | | | Cycle 2 Onwards | | | | EOT^{c} | FU^{d} | FU^{e} | FU^{f} |
| Week | | | | | | | | | | | | | | |
| −4 | | | | | | | | | | | | | | |
| to −1 | 1 | | 2 | 3 | | 4 | 5 | 6 | 7 | 8 | — | — | — | — |
| Cycle Day (±Visit Window^{g}) | | | | | | | | | | | | | | |
| −28 | 1^{h} | 2 | 8 | 15 | 16 | 22 | 1 | 8 | 15 | 22 | | | | |
| to −1 | (±2) | (±2) | (±2) | (±2) | (±2) | (±2) | (±2) | (±2) | (±2) | (±2) | — | — | — | — |

Dosing

| | | | | | | | | |
|---|---|---|---|---|---|---|---|---|
| BT7480 infusionⁱ | X | X | X | X | X | X | X | X |

Procedures

| | | | | | | | | | | | | | |
|---|---|---|---|---|---|---|---|---|---|---|---|---|---|
| Informed consent | X | | | | | | | | | | | | |
| Medical and disease history | X | | | | | | | | | | | | |
| (including smoking history) and | | | | | | | | | | | | | |
| demographics | | | | | | | | | | | | | |
| Inclusion/exclusion criteria | X | | | | | | | | | | | | |
| Full physical examination^{j} | X | X | | | | | | | | | | | |
| Abbreviated physical | | X | X | X | X | X | X | X | X | X | X | X | X |
| examination^{j} | | | | | | | | | | | | | |
| Body weight, height, BMI^{l} | X | X | | | | | | X | | | | X | X |
| Vital signs^{m} | X | X | X | X | X | X | X | X | X | X | X | X | X |
| ECOG performance status | X | X | | | X | | | X | | | | X | X |
| 12-lead ECGⁿ | X | X | | | X | | | X | | | | | |

| | |
|---|---|
| Prior/concomitant medications^{o} | ←---------------------------------------------------------X--------------------------------------------------→ |
| AEs^{p} | ←---------------------------------------------------------X--------------------------------------------------→ |

Laboratory assessments

| Hematology, coagulation^{q} | X | X | | X | X | | X | X | X | X | X | X | X |
|---|---|---|---|---|---|---|---|---|---|---|---|---|---|
| Chemistry^{r} | X | X | X | X | X | X | X | X | X | X | X | X | X |
| Urinalysis^{s} | X | X | | X | X | | X | X | X | X | X | X | X |
| Pregnancy test (for WOCBP)^{t} | X | X | | | | | | X | | | | | X |
| FSH (if postmenopausal) | X | | | | | | | | | | | | |
| Viral testing and serology^{u} | X | | | | | | | | | | | | |
| Sampling for PK^{v} | | [X] | [X] | [X] | [X] | [X] | | [X] | | [X] | | [X] | |
| Blood samples for biomarkers^{w} | [X] | [X] | [X] | [X] | [X] | [X] | | [X] | | [X] | | [X] | |
| Baseline tumor tissue (where | X | | | | | | | | | | | | |
| applicable with confirmation of | | | | | | | | | | | | | |
| Nectin-4 expression)^{x} | | | | | | | | | | | | | |
| Optional pre-treatment tumor | X | | | | | | | | | | | | |
| biopsy^{y} | | | | | | | | | | | | | |
| Optional on-treatment tumor | | | ←-------------------------------X----------------------------→ | | | | | | | | | | |
| biopsy^{y} | | | | | | | | | | | | | |

| | |
|---|---|
| Optional normal skin biopsy^{z} | ←----------------------------X-------------------------→ |

Disease assessments

| | | | | | |
|---|---|---|---|---|---|
| Disease assessment per | X | X^{bb} | X | X | |
| RECIST 1.1^{aa} | | | | | |
| Follow-Up | | | | X | X |

Note:
For procedures marked [X], see Table S4 for the sampling schedule.
^{a}Home health visits may be offered to some sites that are unable to accommodate extended post-dose PK and pharmacodynamic sample collection (see Table S4).
^{b}The DLT period is applicable to dose escalation cohorts only.
^{c}The EOT visit must be performed when a patient permanently discontinues BT7480, preferably before new anticancer therapy(ies) are started. This visit should occur within 1 week (±2 days) and no later than 14 days after the last dose of BT7480, and before the safety FU visit. If the patient finishes treatment without having PD, disease assessment by Investigator per RECIST 1.1 must be performed at EOT unless it has been done within the past 4 weeks.
^{d}The safety FU visit must occur 30 (±5) days after the last dose of BT7480.
^{e}Additional FU visits after the safety FU visit will be performed every 8 weeks (±7 days) for the first 12 months and every 12 weeks (±28 days) thereafter until disease progression, death, or another withdrawal criterion is met.
^{f}After disease progression, patients will be followed every 12 weeks (±28 days) for survival until death or for up to 1 year after the last patient is accrued. Patients may be contacted via outpatient visits or by telephone.
^{g}The visit window may be extended (see footnotes aa and bb).
^{h}On C1D1, complete all procedures and laboratory assessments before starting BT7480 infusion. If screening and laboratory assessments are performed within 72 h (or 14 days for PK and biomarker sampling; see Table S4) prior to starting the first dose of BT7480, they do not need to be repeated on C1D1 except for ECOG performance status, an abbreviated physical examination, vital signs, and triplicate 12-lead ECGs.
ⁱAdminister BT7480 as an intravenous infusion over 60 (−5/+15) min (i.e., infusion periods between 55 and 75 min are acceptable), once per week (or as specified by SRC). During C1, the patient must be assessed for infusion site reactions, including vital signs, within 30 minutes before infusion start, during infusion (minimum of every 30 [±10] minutes), at EOI (−5 min), and post-EOI at 30 and 60 (±10) min.
^{j}A full physical examination will include an evaluation of the following: head, ears, nose, throat, cardiovascular, respiratory, gastrointestinal, neurological, dermatological, and musculoskeletal systems and if applicable, the genitourinary system.
^{k}An abbreviated physical examination will include a symptom-based examination per Investigator's discretion. On C1D1, perform an abbreviated physical examination (and not a full physical examination) if screening assessments have been performed within 72 h prior to starting the first dose of BT7480 on C1D1.
^{l}Recalculate BT7480 dose for patients who experience a ≥10% change in body weight. Height is only measured at screening and will be used to calculate BMI.
^{m}Assess vital signs (systolic and diastolic blood pressure, heart rate, respiration rate, and temperature) after the patient has rested in the sitting position for at least 5 min.
ⁿObtain 12-lead ECGs (for safety monitoring) before other procedures and after the patient has rested for at least 3 min and assess locally. In addition, screening, C1D1, and C1D15 tracings will be done in triplicate by a central ECG vendor and stored for possible future analysis (Phase 1 dose escalation cohorts only). During Cycle 1, ECGs are done on D 1 and D 15 at pre-dose, EOI, and post-EOI at 60 min at same time as plasma PK sampling. During Cycle 2 onwards, ECGs are done on D 1 pre-dose and EOI at a same time as plasma PK sampling. When in triplicate, each recording will be separated by approximately 1 min such that the 3 ECGs are collected within a 5-min window. Safety ECGs must be performed regardless of whether triplicate ECGs are collected.
^{o}Prior medications will be recorded at screening only. Concomitant medications may be given as medically indicated. Any medication taken by the patient during the study, from the time of informed consent until the safety FU visit, will be considered a concomitant medication. All concomitant medications must be recorded in the patient's eCRF along with the reason for use, dates of administration, and dosages.
^{p}All AEs, regardless of causality or seriousness, will be recorded from the time of informed consent until 30 (±5) days after the last dose of BT7480. The Investigator must continue to follow the patient until resolution/stabilization of any ongoing study drug-related AEs or until the condition becomes chronic in nature.
^{q}After C4, schedule CBC with differential counts on D 1 and D 15 only.
^{r}Review C1D1 chemistry results prior to dosing. After C4, schedule chemistry on D 1 and D 15 only.
^{s}Perform a full urinalysis at screening, C1 visits, and EOT; may substitute dipstick with reflex sedimentation at all other visits.
^{t}Perform pregnancy test for WOCBP only at screening (serum), at the beginning of each treatment cycle starting with C1 (urine for C1, and urine or serum for subsequent cycles), and at safety FU (urine or serum).
^{u}Viral testing and serology markers.
^{v}See Table S4 for details on PK blood sampling and PK urine sampling by the hour. When a planned BT7480 dose is held due to an AE and administered on a later day in the week, a pre-treatment PK sample should be collected on the actual dosing date and labeled as ‘unscheduled’.
^{w}See Table S4 for details on sample collection for biomarkers planned for this study.
^{x}Provision of baseline tumor tissue (10 to 15 unstained, FFPE slides) is mandatory. Tumoral Nectin-4 expression will be retrospectively analyzed for all patients using archival tumor tissue or fresh tumor biopsy collected during screening. Archival tumor tissue should be provided as a tissue block or as 10 to 15 FFPE unstained slides.
^{y}During Phase 1 monotherapy (dose escalation [Cohort 3 onwards] and expansion), patients may provide optional paired (pre- and on-treatment) tumor biopsies. If the patient has consented to optional paired tumor biopsies, both a pre-treatment baseline tumor biopsy and archival tumor tissue are requested. On-treatment biopsy sampling is allowed at any time after C1D1 and, if applicable and possible, at relapse. During tumor biopsy collection, collect sufficient cores (≥3) to provide material for a single frozen PK sample and an FFPE block yielding at least 10 to 15 slides. If collection of tumor tissue using a core needle biopsy is not possible, fine needle aspirate samples will be acceptable if sufficient samples (≥5) are collected.
^{z}For patients with an accessible tumor lesion that is feasible to biopsy and has consented to additional biopsies, perform an on-treatment tumor biopsy and normal skin biopsy.
^{aa}For disease assessment by Investigator per RECIST 1.1, perform a CT or MRI scan, or manual measurement of visual lesions or other types of imaging at screening; every 8 weeks (±7 days) for first 12 months, and every 12 weeks (±28 days) thereafter until disease progression, death, or another response withdrawal criterion is met.
^{bb}Starting with C3, perform disease assessment by Investigator per RECIST 1.1 at the start of every odd cycle (±7 days) through 12 months, then every 12 weeks (±28 days) thereafter until disease progression, death, or another response withdrawal criterion is met.
AE = adverse event; BMI = body mass index; C = Cycle; CBC = complete blood count; CT = computed tomography; D = Cycle Day; DLT = dose-limiting toxicity; ECG = electrocardiogram; ECOG = Eastern Cooperative Oncology Group; eCRF = electronic case report form; EOI = end of infusion; EOT = end of treatment; FFPE = formalin-fixed paraffin-embedded; FSH = follicle-stimulating hormone; FU = follow-up; h = hour(s); min = minute(s); MRI = magnetic resonance imaging; OS = overall survival; PD = progressive disease; PFS = progression-free survival; PK = pharmacokinetic(s); RECIST = Response Evaluation Criteria in Solid Tumors; screen = screening; SRC = Safety Review Committee; WOCBP = women of childbearing potential.

TABLE S2
BT7480 Monotherapy - Dose Confirmation Renal Impairment Cohort (Optional) - Phase 1

| | Cycle 1^{a} | | | | | | | | | | | Safety | PFS | OS |
|---|---|---|---|---|---|---|---|---|---|---|---|---|---|---|
| Screen | (DLT Period = 28 Days) | | | | | | Cycle 2 Onwards | | | | EOT^{b} | FU^{c} | FU^{d} | FU^{e} |
| Week | | | | | | | | | | | | | | |
| −4 | | | | | | | | | | | | | | |
| to −1 | 1 | | 2 | 3 | | 4 | 5 | 6 | 7 | 8 | — | — | — | — |
| Cycle Day (±Visit Window^{f}) | | | | | | | | | | | | | | |
| −28 | 1^{g} | 2 | 8 | 15 | 16 | 22 | 1 | 8 | 15 | 22 | | | | |
| to − 1 | (±2) | (±2) | (±2) | (±2) | (±2) | (±2) | (±2) | (±2) | (±2) | (±2) | — | — | — | — |

Dosing

| | | | | | | | | |
|---|---|---|---|---|---|---|---|---|
| BT7480 infusion^{h} | X | X | X | X | X | X | X | X |

Procedures

| | | | | | | | | | | | | | |
|---|---|---|---|---|---|---|---|---|---|---|---|---|---|
| Informed consent | X | | | | | | | | | | | | |
| Medical and disease history | X | | | | | | | | | | | | |
| (including smoking history) and | | | | | | | | | | | | | |
| demographics | | | | | | | | | | | | | |
| Inclusion/exclusion criteria | X | | | | | | | | | | | | |
| Full physical examinationⁱ | X | X | | | | | | | | | | | |
| Abbreviated physical | | X | X | X | X | X | X | X | X | X | X | X | X |
| examination^{j} | | | | | | | | | | | | | |
| Body weight, height, BMI^{k} | X | X | | | | | | X | | | | X | X |
| Vital signs^{l} | X | X | X | X | X | X | X | X | X | X | X | X | X |
| ECOG performance status | X | X | | | X | | | X | | | | X | X |
| 12-lead ECG^{m} | X | X | | | X | | | X | | | | | |

| Prior/concomitant medicationsⁿ | ←-----------------------------------------------------X----------------------------------------------------→ | |
|---|---|---|
| AEs^{o} | | ←------------------------------------------------X--------------------------------------------→ |

Laboratory assessments

| | | | | | | | | | | | | | |
|---|---|---|---|---|---|---|---|---|---|---|---|---|---|
| Hematology, coagulation^{p} | X | X | | X | X | | X | X | X | X | X | X | X |
| Chemistry, including uric acid^{q} | X | X | X | X | X | X | X | X | X | X | X | X | X |
| Urinalysis^{r} | X | X | | X | X | | X | X | X | X | X | X | X |
| Pregnancy test (for WOCBP)^{s} | X | X | | | | | | X | | | | | X |
| FSH (if postmenopausal) | X | | | | | | | | | | | | |
| Viral testing and serology^{t} | X | | | | | | | | | | | | |
| Hematology, coagulation^{p} | X | X | | X | X | | X | X | X | X | X | X | X |
| Blood samples for biomarkers^{v} | [X] | [X] | [X] | [X] | [X] | [X] | | [X] | | [X] | | [X] | |
| Baseline tumor tissue^{w} | X | | | | | | | | | | | | |
| Optional pre-treatment tumor | X | | | | | | | | | | | | |
| biopsy^{x} | | | | | | | | | | | | | |

| Optional on-treatment tumor biopsy^{x} | ←-------------------------------X------------------------------→ | |
|---|---|---|
| Optional normal skin biopsy^{y} | | ←----------------------------X---------------------------→ |

Disease assessments

| | | | | | |
|---|---|---|---|---|---|
| Disease assessment per RECIST 1.1^{z} | X | X^{aa} | X | X | |
| Follow-Up | | | | X | X |

Note:
For procedures marked [X], see Table S4 for the sampling schedule.
^{a}Home health visits may be offered to some sites that are unable to accommodate extended post-dose PK and pharmacodynamic sample collection (see Table S4).
^{b}The EOT visit must be performed when a patient permanently discontinues BT7480, preferably before new anticancer therapy(ies) are started. This visit should occur within 1 week (±2 days) and no later than 14 days after the last dose of BT7480, and before the safety FU visit. If the patient finishes treatment without having PD, disease assessment by Investigator per RECIST 1.1 must be performed at EOT, unless it has been done within the past 4 weeks.
^{c}The safety FU visit must occur 30 (±5) days after the last dose of BT7480.
^{d}Additional FU visits after the safety FU visit will be performed every 8 weeks (±7 days) for the first 12 months and every 12 weeks (±28 days) thereafter until disease progression, death, or another withdrawal criterion is met.
^{e}After disease progression, patients will be followed every 12 weeks (±28 days) for survival until death or for up to 1 year after the last patient is accrued. Patients may be contacted via outpatient visits or by telephone.
^{f}The visit window may be extended (see footnotes z and aa).
^{g}On C1D1, complete all procedures and laboratory assessments before starting BT7480 infusion. If screening and laboratory assessments are performed within 72 h (or 14 days for PK and biomarker sampling; see Table S4) prior to starting the first dose of BT7480, they do not need to be repeated on C1D1 except for ECOG performance status, an abbreviated physical examination, vital signs, and 12-lead ECGs.
^{h}Administer BT7480 as an intravenous infusion over 60 (−5/+15) min (i.e., infusion periods between 55 and 75 min are acceptable), once per week. During C1, the patient must be assessed for infusion site reactions, including vital signs, within 30 minutes before infusion start, during infusion (minimum of every 30 [±10] minutes), at EOI (−5 min), and post-EOI at 30 and 60 (±10) min.
ⁱA full physical examination will include an evaluation of the following: head, ears, nose, throat, cardiovascular, respiratory, gastrointestinal, neurological, dermatological, and musculoskeletal systems and if applicable, the genitourinary system.
^{j}An abbreviated physical examination will include a symptom-based examination per Investigator's discretion. On C1D1, perform an abbreviated physical examination (and not a full physical examination) if screening assessments have been performed within 72 h prior to starting the first dose of BT7480 on C1D1.
^{k}Recalculate BT7480 dose for patients who experience a ≥10% change in body weight. Height is only measured at screening and will be used to calculate BMI.
^{l}Assess vital signs (systolic and diastolic blood pressure, heart rate, respiration rate, and temperature) after the patient has rested in the sitting position for at least 5 min.
^{m}Obtain 12-lead ECGs (for safety monitoring) before other procedures and after the patient has rested for at least 3 min and assess locally. During Cycle 1, ECGs are done on D 1 and D 15 at pre-dose, EOI, and post-EOI at 60 min at same time as plasma PK sampling. During Cycle 2 onwards, ECGs are done on D 1 pre-dose and EOI at a same time as plasma PK sampling.
ⁿPrior medications will be recorded at screening only. Concomitant medications may be given as medically indicated. Any medication taken by the patient during the study, from the time of informed consent until the safety FU visit, will be considered a concomitant medication. All concomitant medications must be recorded in the patient's eCRF along with the reason for use, dates of administration, and dosages.
^{o}All AEs, regardless of causality or seriousness, will be recorded from the time of informed consent until 30 (±5) days after the last dose of BT7480. The Investigator must continue to follow the patient until resolution/stabilization of any ongoing study drug-related AEs or until the condition becomes chronic in nature.
^{p}After C4, schedule CBC with differential counts on D 1 and D 15 only.
^{q}Review C1D1 chemistry results prior to dosing. After C4, schedule chemistry on D 1 and D 15 only.
^{r}Perform a full urinalysis at screening, C1 visits, and EOT; may substitute dipstick with reflex sedimentation at all other visits.
^{s}Perform pregnancy test for WOCBP only at screening (serum), at the beginning of each treatment cycle starting with C1 (urine for C1 and urine or serum for any subsequent cycles), and at safety FU (urine or serum).
^{t}Viral testing and serology markers.
u. See Table S4 for details on PK blood sampling and PK urine sampling by the hour. When a planned BT7480 dose is held due to an AE and administered on a later day in the week, a pre-treatment PK sample should be collected on the actual dosing date and labeled as ‘unscheduled’.
^{v}See Table S4 for details on sample collection for biomarkers planned for this study.
^{w}Provision of baseline tumor tissue (10 to 15 unstained, FFPE slides) is mandatory. Tumoral Nectin-4 expression will be retrospectively analyzed for all patients using archival tumor tissue or fresh tumor biopsy collected during screening. Archival tumor tissue should be provided as a tissue block or as 10 to 15 FFPE unstained slides.
^{x}Patients in renal impairment cohorts may provide optional paired (pre- and on-treatment) tumor biopsies If the patient has consented to optional paired tumor biopsies, both a pre-treatment baseline tumor biopsy and archival tumor tissue are requested. On-treatment biopsy sampling is allowed at any time after C1D1 and, if applicable and possible, at relapse. During tumor biopsy collection, collect sufficient cores (≥3) to provide material for a single frozen PK sample and an FFPE block yielding at least 10 to 15 slides. If collection of tumor tissue using a core needle biopsy is not possible, fine needle aspirate samples will be acceptable if sufficient samples (≥5) are collected.
^{y}For patients with an accessible tumor lesion that is feasible to biopsy and has consented to additional biopsies, perform an on-treatment tumor biopsy and normal skin biopsy.
^{z}For disease assessment by Investigator per RECIST 1.1, perform a CT or MRI scan, or manual measurement of visual lesions or other types of imaging at screening; every 8 weeks (±7 days) for first 12 months; and every 12 weeks (±28 days) thereafter until disease progression, death, or another response withdrawal criterion is met.
^{aa}Starting with C3, perform disease assessment by Investigator per RECIST 1.1 at the start of every odd cycle (±7 days) through 12 months, then every 12 weeks (±28 days) thereafter until disease progression, death, or another response withdrawal criterion is met.
AE = adverse event; BMI = body mass index; C = Cycle; CBC = complete blood count; CT = computed tomography; D = Cycle Day; DLT = dose-limiting toxicity; ECG = electrocardiogram; ECOG = Eastern Cooperative Oncology Group; eCRF = electronic case report form; EOI = end of infusion; EOT = end of treatment; FFPE = formalin-fixed paraffin-embedded; FSH = follicle-stimulating hormone; FU = follow-up; h = hour(s); min = minute(s); MRI = magnetic resonance imaging; OS = overall survival; PD = progressive disease; PFS = progression-free survival; PK = pharmacokinetic(s); RECIST = Response Evaluation Criteria in Solid Tumors; screen = screening; WOCBP = women of childbearing potential.

TABLE S3
Combination Therapy (Optional) - Dose Escalation and Dose Expansion Cohorts - Phase 1 and Phase 2

| | Cycle 1^{a} | | | | | | | | | | | Safety | PFS | OS |
|---|---|---|---|---|---|---|---|---|---|---|---|---|---|---|
| Screen | (DLT Period = 28 Days)^{b} | | | | | | Cycle 2 Onwards | | | | EOT^{c} | FU^{d} | FU^{e} | FU^{f} |
| Week | | | | | | | | | | | | | | |
| −4 | | | | | | | | | | | | | | |
| to −1 | 1 | | 2 | 3 | | 4 | 5 | 6 | 7 | 8 | — | — | — | — |
| Cycle Day (±Visit Window^{g}) | | | | | | | | | | | | | | |
| −28 | 1^{h} | 2 | 8 | 15 | 16 | 22 | 1 | 8 | 15 | 22 | | | | |
| to −1 | (±2) | (±2) | (±2) | (±2) | (±2) | (±2) | (±2) | (±2) | (±2) | (±2) | — | — | — | — |

Dosing

| | | | | | | | | |
|---|---|---|---|---|---|---|---|---|
| BT7480 infusion^{i,j} | X | X | X | X | X | X | X | X |

Procedures

| | | | | | | | | | | | | | |
|---|---|---|---|---|---|---|---|---|---|---|---|---|---|
| Nivolumab infusion^{j} | | X | | | X | | | X | | X | | | |
| Informed consent | X | | | | | | | | | | | | |
| Medical and disease history | X | | | | | | | | | | | | |
| (including smoking history), and | | | | | | | | | | | | | |
| demographics | | | | | | | | | | | | | |
| Inclusion/exclusion criteria | X | | | | | | | | | | | | |
| Full physical examination^{k} | X | X | | | | | | | | | | | |
| Abbreviated physical | | X | X | X | X | X | X | X | X | X | X | X | X |
| examination^{l} | | | | | | | | | | | | | |
| Body weight, height, and BMI^{m} | X | X | | | | | | X | | | | X | X |
| Vital signsⁿ | X | X | X | X | X | X | X | X | X | X | X | X | X |
| ECOG performance status | X | X | | | X | | | X | | | | X | X |
| 12-lead ECG^{o} | X | X | | | X | | | X | | | | | |

| Prior/concomitant medications^{p} | ←-----------------------------------------------------------X---------------------------------------------→ | |
|---|---|---|
| AEs^{q} | | ←------------------------------------------------X--------------------------------------------→ |

Laboratory assessments

| | | | | | | | | | | | | | |
|---|---|---|---|---|---|---|---|---|---|---|---|---|---|
| Hematology, coagulation^{r} | X | X | | X | X | | X | X | X | X | X | X | X |
| Chemistry^{s} | X | X | X | X | X | X | X | X | X | X | X | X | X |
| Autoimmune and endocrine tests^{t} | X | | | | | | | X | | | | X | |
| Urinalysis^{u} | X | X | | X | X | | X | X | X | X | X | X | X |
| Pregnancy test (for WOCBP)^{v} | X | X | | | | | | X | | | | | X |
| FSH (if postmenopausal) | X | | | | | | | | | | | | |
| Viral testing and serology^{w} | X | | | | | | | | | | | | |
| Samples for PK^{x} | | [X] | [X] | [X] | [X] | [X] | | [X] | | [X] | | [X] | |
| Blood samples for biomarkers^{y} | [X] | [X] | [X] | [X] | [X] | [X] | | [X] | | [X] | | [X] | |
| Baseline tumor tissue (where | X | | | | | | | | | | | | |
| applicable with confirmation of | | | | | | | | | | | | | |
| Nectin-4 expression)^{z} | | | | | | | | | | | | | |
| Optional pre-treatment tumor | X | | | | | | | | | | | | |
| biopsy^{aa} | | | | | | | | | | | | | |

| Optional on-treatment tumor | ←----------------------------X---------------------------------→ | |
|---|---|---|
| biopsy^{aa} | | |
| Optional normal skin biopsy^{bb} | | ←-------------------------X---------------------------→ |

Disease assessments

| | | | | | | |
|---|---|---|---|---|---|---|
| Disease assessment per | X | X^{dd} | X | X | X | |
| RECIST 1.1^{cc} | | | | | | |
| Follow-Up | | | | | X | X |

Note:
For procedures marked [X], see Table S4 for the sampling schedule.
Note:
For combination therapy, EOI refers to the end of BT7480 infusion.
^{a}Home health visits may be offered to some sites that are unable to accommodate extended post-dose PK and pharmacodynamic sample collection (see Table S4).
^{b}The DLT period is applicable for dose escalation cohorts only.
^{c}The EOT visit must be performed when a patient permanently discontinues study drug (BT7480 and/or nivolumab), preferably before new anticancer therapy(ies) are started. This visit should occur within 1 week (±2 days) and no later than 14 days after the last dose of study drug, and before the safety FU visit. If the patient finishes treatment without having PD, disease assessment by Investigator per RECIST 1.1 must be performed at EOT, unless it has been done within the past 4 weeks.
^{d}The safety FU must occur 125 (±5) days after the last dose of study drug.
^{e}Additional FU visits after the safety FU visit will be performed every 8 weeks (±7 days) for the first 12 months and every 12 weeks (±28 days) thereafter until disease progression, death, or another withdrawal criterion is met.
^{f}After disease progression, patients will be followed every 12 weeks (±28 days) for survival until death or for up to 1 year after the last patient is accrued. Patients may be contacted via outpatient visits or by telephone.
^{g}The visit window may be extended (see footnotes cc and dd).
^{h}On C1D1, complete all procedures and laboratory assessments before starting BT7480 infusion. If screening and laboratory assessments are performed within 72 h (or 14 days for PK and biomarker sampling; see Table S4) prior to starting the first dose of BT7480, they do not need to be repeated on C1D1 except for ECOG performance status, an abbreviated physical examination, vital signs, and triplicate 12-lead ECGs.
ⁱAdminister BT7480 as an intravenous infusion over 60 (−5/+15) min (i.e., infusion periods between 55 and 75 min are acceptable), once per week (or as specified by SRC). During C1, the patient must be assessed for infusion site reactions, including vital signs, within 30 minutes before infusion start, during infusion (minimum of every 30 [±10] minutes), at EOI (−5 min), and post-EOI at 30 and 60 (±10) min.
^{j}On nivolumab-coincident dosing days, administer BT7480 first, observe for at least an hour, and then administer nivolumab 240 mg as an intravenous infusion (after proper flushing of the initial line or via a separate line) over 30 (−5/+15) min once every 2 weeks as per the USPI, SmPC, or other local labeling. After completion of nivolumab infusion, observe the patient for at least 30 min or per local labeling or institutional practices for AEs and infusion-related reactions. The total duration of nivolumab administration will be based on patient response to treatment and Investigator judgment.
^{k}A full physical examination will include an evaluation of the following: head, ears, nose, throat, cardiovascular, respiratory, gastrointestinal, neurological, dermatological, and musculoskeletal systems and if applicable, the genitourinary system.
^{l}An abbreviated physical examination will include a symptom-based examination per Investigator's discretion. On C1D1, perform an abbreviated physical examination (and not a full physical examination) if screening assessments have been performed within 72 h prior to starting the first dose of BT7480 on C1D1.
^{m}Recalculate BT7480 dose for patients who experience a ≥10% change in body weight. Height is only measured at screening and will be used to calculate BMI.
ⁿAssess vital signs (systolic and diastolic blood pressure, heart rate, respiration rate, and temperature) after the patient has rested in the sitting position for at least 5 min.
^{o}Obtain 12-lead ECGs (for safety monitoring) before other procedures and after the patient has rested for at least 3 min and assess locally. In addition, screening, C1D1, and C1D15 tracings will be done in triplicate by a central ECG vendor and stored for possible future analysis (Phase 1 dose escalation cohorts only). During Cycle 1, ECGs are done on D 1 and D 15 at pre-dose, EOI, and post-EOI at 60 min at same time as plasma PK sampling. During Cycle 2 onwards, ECGs are done on D 1 pre-dose and EOI at a same time as plasma PK sampling. When in triplicate, each recording will be separated by approximately 1 min such that the 3 ECGs are collected within a 5-min window. Safety ECGs must be performed regardless of whether triplicate ECGs are collected.
^{p}Prior medications will be recorded at screening only. Concomitant medications may be given as medically indicated. Any medication taken by the patient during the study, from the time of informed consent until the safety FU visit, will be considered a concomitant medication. All concomitant medications must be recorded in the patient's eCRF along with the reason for use, dates of administration, and dosages.
^{q}All AEs, regardless of causality or seriousness, will be recorded from the time of informed consent until 125 (±5) days after the last dose of nivolumab. The Investigator must continue to follow the patient until resolution/stabilization of any ongoing study drug-related AEs or until the condition becomes chronic in nature.
^{r}After C4, schedule CBC with differential counts on D 1 and D 15 only.
^{s}Review C1D1 chemistry results prior to dosing. After C4, schedule chemistry on D 1 and D 15 only.
^{t}For patients receiving nivolumab, collect blood samples for the assessment of antinuclear antibody, rheumatoid factor, adrenocorticotrophic hormone, free T3, free T4, and TSH at screening, C2D1, and every 6 months thereafter until disease progression, death, or another withdrawal criterion is met.
^{u}Perform a full urinalysis at screening, C1 visits, and EOT; may substitute dipstick with reflex sedimentation at all other visits.
^{v}Perform pregnancy test for WOCBP only at screening (serum), at the beginning of each treatment cycle starting with C1 (urine for C1, and urine or serum for subsequent cycles), and at safety FU (urine or serum).
^{w}Viral testing and serology markers.
^{x}See Table S4 for details on PK blood sampling and PK urine sampling by the hour. When a planned BT7480 dose is held due to an AE and administered on a later day in the week, a pre-treatment PK sample should be collected on the actual dosing date and labeled as ‘unscheduled’.
^{y}See Table S4 for the blood collection chart for biomarkers planned for this study.
^{z}Provision of baseline tumor tissue (10 to 15 unstained, FFPE slides) is mandatory. Tumoral Nectin-4 expression will be retrospectively analyzed for all patients using archival tumor tissue or fresh tumor biopsy collected during screening. Archival tumor tissue should be provided as a tissue block or as 10 to 15 FFPE unstained slides.
^{aa}During Phase 1 combination therapy (dose escalation and expansion), patients may provide optional paired (pre- and on-treatment) tumor biopsies. If the patient has consented to optional paired tumor biopsies, both a pre-treatment baseline tumor biopsy and archival tumor tissue are requested. On-treatment biopsy sampling is allowed at any time after C1D1 and, if applicable and possible, at relapse. During tumor biopsy collection, collect sufficient cores (≥3) to provide material for a single frozen PK sample and an FFPE block yielding at least 10 to 15 slides. If collection of tumor tissue using a core needle biopsy is not possible, fine needle aspirate samples will be acceptable if sufficient samples (≥5) are collected.
^{bb}For patients with an accessible tumor lesion that is feasible to biopsy and has consented to additional biopsies, perform an on-treatment tumor biopsy and normal skin biopsy.
^{cc}For disease assessment by Investigator per RECIST 1.1, perform a CT or MRI scan, or manual measurement of visual lesions or other types of imaging at screening; every 8 weeks (±7 days) for first 12 months; and every 12 weeks (±28 days) thereafter until disease progression, death, or another response withdrawal criterion is met.
^{dd}Starting with C3, perform disease assessment by Investigator per RECIST 1.1 at the start of every odd cycle (±7 days) through 12 months, then every 12 weeks (±28 days) thereafter until disease progression, death, or another response withdrawal criterion is met.
AE = adverse event; BMI = body mass index; C = Cycle; CBC = complete blood count; CT = computed tomography; D = Cycle Day; DLT = dose-limiting toxicity; ECG = electrocardiogram; ECOG = Eastern Cooperative Oncology Group; eCRF = electronic case report form; EOI = end of infusion; EOT = end of treatment; FFPE = formalin-fixed paraffin-embedded; FSH = follicle-stimulating hormone; FU = follow-up; h = hour(s); min = minute(s); MRI = magnetic resonance imaging; OS = overall survival; PD = progressive disease; PFS = progression-free survival; PK = pharmacokinetic(s); RECIST = Response Evaluation Criteria in Solid Tumors; screen = screening; SmPC = Summary of Product Characteristics; SOI = start of infusion; SRC = Safety Review Committee; T3 = triiodothyronine; T4 = thyroxine; TSH = thyroid-stimulating hormone; USPI = United States Prescribing Information; WOCBP = women of childbearing potential.

TABLE S4
Peripheral Biomarker Sample Collection Chart - Monotherapy and Combination Therapy - Phase 1 and Phase 2

| | Scr | Cycle 1 | | | | | | | | | |
|---|---|---|---|---|---|---|---|---|---|---|---|
| | Cycle Day | | | | | | | | | | |
| | −28 to −1 | 1 and 15 | | | | | | | | | |
| | Collection Time^{a} | | | | | | | | | | |
| | | | Post-SOI | Pre-EOI^{b,c} | Post-EOI^{b} | | | | | | |
| | — | Pre-SOI | 30 m | −5 m | 10 m | 20 m | 30 m | 1 h | 3 h | 4 h | 6 h |
| | Collection Window (clinical site visits) | | | | | | | | | | |
| | — | — | ±5 m | ±2 m | ±5 m | ±5 m | ±5 m | ±5 m | ±10 m | ±10 m | ±10 m |
| | Collection Window (home health visits)^{d} | | | | | | | | | | |
| | — | — | — | — | — | — | — | — | — | — | — |
| Phase 1 PK blood | | X | X | X^{c} | X | X | X | X | X | X | X |
| sampling | | | | | | | | | | | |
| Phase 2 PK blood | | X | X | X^{c} | | | | | | | X |
| sampling | | | | | | | | | | | |
| Phase 1 PK urine | | | ←--------------------------X^{e}-------------------------→ | | | | | | | | |
| sampling | | | | | | | | | | | |
| ADAs | | X | | | | | | | | | |
| CD137 RO | X | X | | | | X | | | X | | |
| Circ. mech. proteins | | X | | | | | | | | | X |
| Inflammatory | X | X | | | | | | | | | X |
| cytokines^{h} | | | | | | | | | | | |
| NAbs | | X | | | | | | | | | |
| Immunophenotyping | X | X | | | | | | | | | |
| RNA immune | | X | | | | | | | | | |
| profiling | | | | | | | | | | | |
| ctDNA PGx | | X | | | | | | | | | |
| Germline genomics | | Xⁱ | | | | | | | | | |

| | Cycle 1 | | | | | Cycle 2 Onwards | | | |
|---|---|---|---|---|---|---|---|---|---|
| | Cycle Day | | | | | | | | |
| | 1 and 15 | | | | 8 | 1 | | 15 | EOT |
| | Collection Time^{a} | | | | | | | | |
| | Post-EOI^{b} | | | | | | Pre-EOI^{b,c} | | |
| | 24 h | 48 h | 72 h | 96 h | Pre-SOI | Pre-SOI | −5 m | Pre-SOI | |
| | Collection Window (clinical site visits) | | | | | | | | |
| | ±1 h | ±1 h | ±1 h | ±1 h | — | — | | | — |
| | Collection Window (home health visits)^{d} | | | | | | | | |
| | — | ±12 h | ±12 h | ±12 h | — | — | | | — |
| Phase 1 PK blood | X | X | X | X | X | X | X^{c} | X | X |
| sampling | | | | | | | | | |
| Phase 2 PK blood | | X | X | | | X | X^{c} | | |
| sampling | | | | | | | | | |
| Phase 1 PK urine | X | X | X | X | X | | | | |
| sampling | | | | | | | | | |
| ADAs | | | | | | X | | | X |
| CD137 RO | ←----X^{f}---→ | | | | | X^{g} | | | X |
| Circ. mech. proteins | ←----X^{f}---→ | | | | X | X | | X | X |
| Inflammatory | ←----X^{f}---→ | | | | | X | | X | X |
| cytokines^{h} | | | | | | | | | |
| NAbs | | | | | | X | | | X |
| Immunophenotyping | | | | | X | X | | | X |

| | | | | |
|---|---|---|---|---|
| RNA immune | ←----X^{f}---→ | X | X | X |
| profiling | | | | |
| ctDNA PGx | | | X | X |
| Germline genomics | | | | |

^{a}The exact timing of sample collection must be captured on the appropriate electronic case report form and requisition page(s).
^{b}For combination therapy, EOI refers to the end of BT7480 infusion.
^{c}Draw blood sample for PK analyses just prior to end of BT7480 infusion.
^{d}Home health visits may be offered to some sites that are unable to accommodate sample collection at 48 (±12), 72 (±12), and 96 (±12) h post-EOI on C1D1 and C1D15.
^{e}Collect urine from SOI to 6 h post-EOI and record the total voided volume. Total voided volume is not required at 24, 48, 72, and 96 h.
^{f}Collect sample once between 24 (±3) to 72 (±12) h post-EOI at the time of PK blood sampling.
^{g}Collect sample on C2D1 only.
^{h}Blood sample should also be collected immediately in the event an infusion-related reaction or suspected cytokine release syndrome is observed.
ⁱCollect sample on C1D1 only.
ADA = anti-drug antibody; C = Cycle; CD = cluster of differentiation; circ. = circulating; ctDNA = circulating tumor deoxyribonucleic acid; D = Cycle Day; EOI = end of infusion; EOT = end of treatment; h = hour(s); m = minute(s); mech. = mechanistic; NAb = neutralizing antibody; PGx = pharmacogenomic(s); PK = pharmacokinetic(s); RNA = ribonucleic acid; RO = receptor occupancy; scr = screening; SOI = start of infusion.

1. A solid pharmaceutical composition comprising BT7480, or a pharmaceutically acceptable salt thereof, Tris base, mannitol, and sodium hydroxide.

2. The solid pharmaceutical composition of claim 1, comprising about 0.1 mg Tris base per mg of BT7480, or a pharmaceutically acceptable thereof.

3. The solid pharmaceutical composition of claim 1 or 2, comprising about 0.4 mg mannitol per mg of BT7480, or a pharmaceutically acceptable thereof.

4. The solid pharmaceutical composition of any one of claims 1-3, wherein the amount of sodium hydroxide provides a pH of about 7.0 to 7.5 when the solid pharmaceutical composition is reconstituted in water.

5. The solid pharmaceutical composition of any one of claims 1-4, comprising:
about 66 mg BT7480, or a pharmaceutically acceptable salt thereof;
about 6.6 mg Tris base;
about 26.4 mg mannitol; and
sodium hydroxide, wherein the amount of sodium hydroxide provides a pH of about 7.0 to 7.5 when the solid pharmaceutical composition is reconstituted in water.

6. An aqueous pharmaceutical composition comprising BT7480, or a pharmaceutically acceptable salt thereof, Tris base, mannitol, sodium hydroxide, and water.

7. The aqueous pharmaceutical composition of claim 6, comprising about 60 mg/mL BT7480, or a pharmaceutically acceptable salt thereof.

8. The aqueous pharmaceutical composition of claim 6 or 7, comprising about 6 mg/mL Tris base.

9. The aqueous pharmaceutical composition of any one of claims 6-8, comprising about 24 mg/mL mannitol.

10. The aqueous pharmaceutical composition of any one of claims 6-9, wherein the aqueous pharmaceutical composition is at a pH of about 7.0 to 7.5.

11. The aqueous pharmaceutical composition of any one of claims 6-10, which is at a volume of about 1.1 mL.

12. The aqueous pharmaceutical composition of any one of claims 6-11, comprising:
about 60 mg/mL BT7480, or a pharmaceutically acceptable salt thereof;
about 6 mg/mL Tris base;
about 24 mg/mL mannitol;
sodium hydroxide; and
water, wherein the aqueous pharmaceutical composition is at a pH of about 7.0 to 7.5.

13. The aqueous pharmaceutical composition of any one of claims 6-12, further comprising sodium chloride.

14. A method for treating a Nectin-4 associated advanced malignancy in a patient comprising intravenously administering to the patient the aqueous pharmaceutical composition of any one of claims 6-13.

15. The method of claim 14, wherein the Nectin-4 associated advanced malignancy is selected from the group consisting of non-small-cell lung cancer (NSCLC), ovarian cancer, triple-negative breast cancer (TNBC), gastric/upper gastrointestinal (GI) cancer, pancreatic cancer, urothelial cancer, bladder cancer, head and neck cancer, esophageal cancer, and melanoma.

16. The method of claim 14 or 15, wherein the aqueous pharmaceutical composition is administered once weekly.

17. The method of any one of claims 14-16, wherein the aqueous pharmaceutical composition is administered at a dose of up to about 7.5 mg/kg.

18. The method of any one of claims 14-17, wherein the aqueous pharmaceutical composition is administered via an IV infusion of about 60 minutes.

19. The method of any one of claims 14-18, further comprising administering Nivolumab.

20. The method of claim 19, wherein Nivolumab is administered via an IV infusion of about 30 minutes.
